(19) United States
(12) Patent Application Publication
(10) Pub. No.: US 2007/0275929 A1
(43) Pub. Date: Nov. 29, 2007

(54) Composition and method for controlling the transmission of noroviruses

(75) Inventors: Janice L. Fuls, Fountain Hills, AZ (US); Nancy D. Rodgers, Chandler, AZ (US); Richard F. Theiler, Scottsdale, AZ (US); Daniel E. Pedersen, Cottage Grove, MN (US); Katherine J. Molinaro, Inver Grove Heights, MN (US); John J. Rolando, Woodbury, MN (US)

Correspondence Address: MARSHALL, GERSTEIN & BORUN LLP (DIAL), 233 S. WACKER DRIVE, 6300 SEARS TOWER, CHICAGO, IL, 60606, US

(73) Assignee: THE DIAL CORPORATION, Scottsdale, AZ (US)

(21) Appl. No.: 11/805,296

(22) Filed: May 23, 2007

Related U.S. Application Data
(60) Provisional application No. 60/811,354, filed on Jun. 6, 2006.
(60) Provisional application No. 60/802,911, filed on May 24, 2006.

(51) Int. Cl. A61K 31/66; A61K 31/185; A61K 31/19; A61K 31/045

(52) U.S. Cl. 514/75; 514/553; 514/724; 514/557; 514/574

(57) ABSTRACT

Antimicrobial compositions having a rapid effectiveness against noroviruses and against bacteria are disclosed. The antimicrobial compositions contain a disinfecting alcohol, an organic acid, and water, wherein the composition has a pH of about 5 or less and the nonvolatile components of the composition are capable of forming a barrier film or layer on a treated surface.

### CROSS-REFERENCE TO RELATED APPLICATIONS

This application claims the benefit of U.S. Provisional Patent Application No. 60/802,911, filed May 24, 2006 and U.S. Provisional Patent Application No. 60/811,354, filed Jun. 6, 2006.

### FIELD OF THE INVENTION

The present invention relates to antimicrobial compositions having a rapid antiviral effectiveness. More particularly, the present invention relates to antimicrobial compositions, such as hand sanitizer compositions, comprising a disinfecting alcohol, an organic acid, and an optional active antimicrobial agent, which are effective in controlling noroviruses. The composition has a pH of about 5 or less, and provides a substantial reduction in norovirus populations within one minute. In some embodiments, compositions provide a barrier layer, or film, of the organic acid on a treated surface to impart a persistent antiviral activity to the surface.

### BACKGROUND OF THE INVENTION

Human health is impacted by a variety of microbes encountered on a daily basis. In particular, contact with various microbes in the environment can lead to an illness, possibly severe, in mammals. For example, microbial contamination can lead to a variety of illnesses, including, but not limited to, food poisoning, a streptococcal infection, anthrax (cutaneous), athlete's foot, cold sores, conjunctivitis (“pink eye”), coxsackievirus (hand-foot-mouth disease), croup, diphtheria (cutaneous), ebolic hemorrhagic fever, and impetigo.

It is known that washing body parts (e.g., hand washing) and hard surfaces (e.g., countertops and sinks) can significantly decrease the population of microorganisms, including pathogens. Therefore, cleaning skin and other animate and inanimate surfaces to reduce microbial populations is a first defense in removing such pathogens from these surfaces, and thereby minimizing the risk of infection.

Viruses are one category of pathogens that are of primary concern. Viral infections are among the greatest causes of human morbidity, with an estimated 60% or more of all episodes of human illness in developed countries resulting from a viral infection. In addition, viruses infect virtually every organism in nature, with high virus infection rates occurring among all mammals, including humans, pets, livestock, and zoo specimens.

Viruses exhibit an extensive diversity in structure and lifecycle. A detailed description of virus families, their structures, life cycles, and modes of viral infection is discussed in Fundamental Virology, 4th Ed., Eds. Knipe & Howley, Lippincott Williams & Wilkins, Philadelphia, Pa., 2001.

Simply stated, virus particles are intrinsic obligate parasites, and have evolved to transfer genetic material between cells and encode sufficient information to ensure their own propagation. In a most basic form, a virus consists of a small segment of nucleic acid encased in a simple protein shell. The broadest distinction between viruses is the enveloped and nonenveloped viruses, i.e., those that do or do not contain, respectively, a lipid-bilayer membrane.

Viruses propagate only within living cells. The principal obstacle encountered by a virus is gaining entry into the cell, which is protected by a cell membrane of thickness comparable to the size of the virus. In order to penetrate a cell, a virus first must become attached to the cell surface. Much of the specificity of a virus for a certain type of cell lies in its ability to attach to the surface of that specific cell. Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell. Therefore, in order to control a viral infection, it is important to rapidly kill a virus that contacts the skin, and ideally to provide a persistent antiviral activity on the skin, or an inanimate surface, in order to control viral infections.

For example, rhinoviruses, influenza viruses, and adenoviruses are known to cause respiratory infections. Rhinoviruses are members of the picornavirus family, which is a family of “naked viruses” that lack an outer envelope. The human rhinoviruses are so termed because of their special adaptation to the nasopharyngeal region, and are the most important etiological agents of the common cold in adults and children. Officially there are 102 rhinovirus serotypes. Most of the picornaviruses isolated from the human respiratory system are acid labile, and this lability has become a defining characteristic of rhinoviruses.

Rhinovirus infections are spread from person to person by direct contact with virus-contaminated respiratory secretions. Typically, this contact is in the form of physical contact with a contaminated surface, rather than via inhalation of airborne viral particles.

Rhinoviruses can survive on environmental surfaces for hours after initial contamination, and infection is readily transmitted by finger-to-finger contact, and by contaminated environmental surface-to-finger contact, if the newly contaminated finger then is used to rub an eye or touch the nasal mucosa. Therefore, virus contamination of skin and environmental surfaces should be minimized to reduce the risk of transmitting the infection to the general population.

Several gastrointestinal infections also are caused by viruses. For example, noroviruses are estimated to cause 23 million cases of acute gastroenteritis in the United States per year, and are the leading cause of gastroenteritis in the United States. Of viruses, only the common cold is reported more often than viral gastroenteritis (norovirus). Norovirus causes nausea, vomiting (sometimes accompanied by diarrhea), and stomach cramps. This infection typically is spread from person to person by direct contact.

Noroviruses (genus Norovirus, family Caliciviridae) are a group of related, single-stranded RNA, nonenveloped viruses that cause acute gastroenteritis in humans. Norovirus recently was approved as the official genus name for the group of viruses provisionally described as “Norwalk-like viruses” (NLV). This group of viruses also has been referred to as calciviruses because of their virus family name, and as small round structured viruses, or SRSVs, because of their morphologic features. Norwalk virus is the prototype virus of the genus Norovirus of the family Caliciviridae. Another genus of the calicivirus family that can cause gastroenteritis in humans is Sapovirus, formerly described as “Sapporo-like virus” (SLV) and sometimes referred to as classic or typical calicivirus.

Noroviruses are genetically classified into five different genogroups (GI, GII, GIII, GIV, and GV), which can be further divided into different genetic groups or genotypes. For example, genogroup II, the most prevalent human genogroup, presently contains 17 genotypes. Genogroups I, II, and IV infect humans. Historically, noroviruses have been named after the place where an outbreak occurs (e.g., Norwalk, Hawaii, Snow Mountain, Southamptom, or Bristol), but recently a numeric classification system has been accepted globally. This classification system is based upon numbering genogroups with Roman numerals and genotypes with numbers. For example, the genogroup II norovirus, Lordsdale virus is a member of genotype 4, and, therefore, classified as a GII.4 norovirus. GII.4 viruses account for the majority of adult outbreaks of gastroenteritis and often are pandemic.

Noroviruses are very highly contagious and can spread easily from person to person. Both stool (feces) and vomit are infectious. It is theorized that an inoculum of as few as 10 viral particles may be sufficient to infect an individual. Noroviruses are transmitted primarily through the fecal-oral route, either by consumption of fecally contaminated food or water, or by direct person-to-person spread. Environmental and fomite contamination also can act as a source of infection.

People can become infected with the norovirus in several ways, including, eating food or drinking liquids that are contaminated with norovirus; touching surfaces or objects contaminated with norovirus, and then placing their hands in their mouths; or having direct contact with another person who is infected and showing symptoms (for example, when caring for someone who is ill, or sharing foods or eating utensils with someone who is ill). During outbreaks of norovirus gastroenteritis, several modes of transmission have been documented, for example, initial foodborne transmission in a restaurant, followed by secondary person-to-person transmission to household contacts. No evidence suggests that norovirus infection occurs through the respiratory system.

Protracted outbreaks of norovirus disease have been reported among elderly persons living in institutional settings, e.g., nursing homes. In some cases, the outbreak was initially caused by exposure to a fecally-contaminated vehicle (e.g., food or water). Then, the outbreak spreads through person-to-person transmission among the residents. This spread is facilitated by the enclosed living quarters and reduced levels of personal hygiene that result from incontinence, immobility, or reduced mental alertness. Because of underlying medical conditions, the disease among these elderly persons can be severe or fatal.

Passengers and crew members on cruise ships and naval vessels are frequently affected by outbreaks of gastroenteritis. Cruise ships often dock in countries where sanitation levels are inadequate, thus increasing the contamination risk of water and food taken aboard or having a passenger board with an active infection. After a passenger or crew member brings the norovirus on board, the close living quarters on ships amplify opportunities for person-to-person transmission. Furthermore, the arrival of new and susceptible passengers every few days or weeks on affected cruise ships provides an opportunity for sustained transmission during successive cruises. Norovirus outbreaks extending beyond twelve successive cruises have been reported.

Currently, no antiviral medication against norovirus is available, and no standard method to prevent infection exists. Norovirus infection cannot be treated with antibiotics. Noroviruses also are relatively resistant to environmental challenge. Noroviruses can survive freezing, temperatures as high as 60° C., and even have been associated with illness after being steamed in shellfish. Moreover, noroviruses can survive in up to 10 ppm chlorine, which is well in excess of chlorine levels routinely present in public water systems. Despite these features, relatively simple measures, such as correct handling of cold foods, frequent handwashing, and paid sick leave, may substantially reduce transmission of noroviruses.

Although interruption of person-to-person transmission can be difficult, frequent handwashing with soap and water is a means of prevention. The recommended procedure is to rub all surfaces of lathered hands together vigorously for at least 10 seconds, then thoroughly rinse the hands under a stream of water, especially after toilet visits or changing diapers, and before eating or preparing food. Because environmental surfaces have been implicated in the transmission of enteric viruses, surfaces that have been soiled should be cleaned with an appropriate antimicrobial product (e.g., 10% solution of household bleach).

Common household phenol/alcohol disinfectants are effective in disinfecting contaminated environmental surfaces, but lack persistent virucidal activity. Hand washing is highly effective in disinfecting contaminated fingers, but again suffers from a lack of persistent activity. These shortcomings illustrate the need for improved virucidal compositions having a persistent activity against viruses, such as noroviruses.

Antimicrobial personal care compositions are known in the art. In particular, antibacterial cleansing compositions, which typically are used to cleanse the skin and to destroy bacteria present on the skin, especially the hands, arms, and face of the user, are well-known commercial products.

Antibacterial compositions are used, for example, in the health care industry, food service industry, meat processing industry, and in the private sector by individual consumers. The widespread use of antibacterial compositions indicates the importance consumers place on controlling bacteria populations on skin. The paradigm for antibacterial compositions is to provide a substantial and broad spectrum reduction in bacterial populations quickly and without adverse side effects associated with toxicity and skin irritation. Such antibacterial compositions are disclosed in U.S. Pat. Nos. 6,107,261 and 6,136,771, each incorporated herein by reference.

One class of antibacterial personal care compositions is the hand sanitizer. This class of compositions is used primarily by medical personnel to disinfect the hands and fingers. The hand sanitizer is applied to, and rubbed into, the hands and fingers, and the composition is allowed to evaporate from the skin.

Hand sanitizers contain a high percentage of an alcohol, like ethanol. At the high percent of alcohol present in the composition, the alcohol itself acts as a disinfectant. In addition, the alcohol quickly evaporates to obviate wiping or rinsing skin treated with the hand sanitizer. Hand sanitizers containing a high percentage of an alcohol, i.e., about 40% or greater by weight of the composition, however, have a tendency to dry and irritate the skin.

Antibacterial cleansing compositions typically contain an active antibacterial agent, a surfactant, and various other ingredients, for example, dyes, fragrances, pH adjusters, thickeners, skin conditioners, and the like, in an aqueous and/or alcoholic carrier. Several different classes of antibacterial agents have been used in antibacterial cleansing compositions. Examples of antibacterial agents include bisguanidines (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxydiphenylether). Antimicrobial compositions based on such antibacterial agents exhibit a wide range of antibacterial activity, ranging from low to high, depending on the microorganism to be controlled and the particular antibacterial composition.

Most commercial antibacterial compositions generally offer a low to moderate antibacterial activity, and no reported antiviral activity. Antibacterial activity is assessed against a broad spectrum of microorganisms, including both Gram positive and Gram negative microorganisms. The log reduction, or alternatively the percent reduction, in bacterial populations provided by the antibacterial composition correlates to antibacterial activity. A 1-3 log reduction is preferred, a log reduction of 3-5 is most preferred, whereas a log reduction of less than 1 is least preferred, for a particular contact time, generally ranging from 15 seconds to 5 minutes. Thus, a highly preferred antibacterial composition exhibits a 3-5 log reduction against a broad spectrum of microorganisms in a short contact time.

Virus control poses a more difficult problem, however. By sufficiently reducing bacterial populations, the risk of bacterial infection is reduced to acceptable levels. Therefore, a rapid antibacterial kill is desired. With respect to viruses, however, not only is a rapid kill desired, but a total antiviral activity also is required. This difference is because merely reducing a virus population is insufficient to reduce infection. For example, in the case of a norovirus, about 10 virus particles can cause an infection. In theory, a single virus can cause infection. Therefore, an essentially total, and persistent, antiviral activity is required, or at least desired, for an effective antiviral cleansing composition.

WO 98/01110 discloses compositions comprising triclosan, surfactants, solvents, chelating agents, thickeners, buffering agents, and water. WO 98/01110 is directed to reducing skin irritation by employing a reduced amount of surfactant.

U.S. Pat. No. 5,635,462 discloses compositions comprising PCMX and selected surfactants. The compositions disclosed therein are devoid of anionic surfactants and nonionic surfactants.

EP 0 505 935 discloses compositions containing PCMX in combination with nonionic and anionic surfactants, particularly nonionic block copolymer surfactants.

WO 95/32705 discloses a mild surfactant combination that can be combined with antibacterial compounds, like triclosan.

WO 95/09605 discloses antibacterial compositions containing anionic surfactants and alkylpolyglycoside surfactants.

WO 98/55096 discloses antimicrobial wipes having a porous sheet impregnated with an antibacterial composition containing an active antimicrobial agent, an anionic surfactant, an acid, and water, wherein the composition has a pH of about 3.0 to about 6.0.

N. A. Allawala et al., J. Amer. Pharm. Assoc.—Sci. Ed., Vol. XLII, no. 5, pp. 267-275 (1953) discusses the antibacterial activity of active antibacterial agents in combination with surfactants.

A. G. Mitchell, J. Pharm. Pharmacol., Vol. 16, pp. 533-537 (1964) discloses compositions containing PCMX and a nonionic surfactant that exhibit antibacterial activity.

With respect to hand sanitizer gels, U.S. Pat. No. 5,776,430 discloses a topical antimicrobial cleaner containing chlorhexidine and an alcohol. The compositions contain about 50% to 60%, by weight, denatured alcohol and about 0.65% to 0.85%, by weight, chlorhexidine. The composition is applied to the skin, scrubbed into the skin, then rinsed from the skin.

European Patent Application 0 604 848 discloses a gel-type hand disinfectant containing an antimicrobial agent, 40% to 90% by weight of an alcohol, and a polymer and a thickening agent in a combined weight of not more than 3% by weight. The gel is rubbed into the hands and allowed to evaporate to provide disinfected hands. As illustrated in EP 0 604 848, the amount and identity of the antibacterial agent is not considered important because the hand sanitizer gels contain a high percentage of an alcohol to provide antibacterial activity. However, to control a norovirus, an alcohol alone requires 30 minutes of contact to reduce norovirus populations by a factor of 3. The disclosed compositions often do not provide immediate sanitization against norovirus and do not provide a persistent antimicrobial efficacy.

In general, hand sanitizer gels typically contain: (a) at least 60% by weight ethanol or a combination of lower alcohols, such as ethanol and isopropanol, (b) water, (c) a gelling polymer, such as a crosslinked polyacrylate material, and (d) other ingredients, such as skin conditioners, fragrances, and the like. Hand sanitizer gels are used by consumers to effectively sanitize the hands, without, or after, washing with soap and water, by rubbing the hand sanitizer gel on the surface of the hands. Current commercial hand sanitizer gels rely on high levels of alcohol for disinfection and evaporation, and thus suffer from disadvantages. Specifically, current hand sanitizer gels have a tendency to dry and irritate the skin because of the high levels of alcohol employed in the compositions. Also, because of the volatility of ethanol, the primary active disinfectant does not remain on the skin sufficiently long after application to control noroviruses, thus failing to provide an antimicrobial effect against these viruses.

At alcohol concentrations below 60%, ethanol is not recognized as an antiseptic. Thus, in compositions containing less than 60% alcohol, an additional antimicrobial compound must be present to provide antimicrobial activity. Prior disclosures, however, have not addressed the issue of which composition ingredient in such an antimicrobial composition provides microbe control. Therefore, for formulations containing a reduced alcohol concentration, the selection of an antimicrobial agent that provides both a rapid antimicrobial effect and a persistent antimicrobial benefit is difficult.

U.S. Pat. Nos. 6,107,261 and 6,136,771 disclose highly effective antibacterial compositions. These patents disclose compositions that solve the problem of controlling bacteria on skin and hard surfaces, but are silent with respect to controlling viruses. Applicants are aware of no reference that provides a solution for combating bacteria in a highly effective way, while simultaneously controlling noroviruses, in the form of a single composition.

U.S. Pat. Nos. 5,968,539; 6,106,851; and 6,113,933 disclose antibacterial compositions having a pH of about 3 to about 6. The compositions contain an antibacterial agent, an anionic surfactant, and a proton donor.

Antiviral compositions disclosed as inactivating or destroying pathogenic viruses, including rhinovirus, rotavirus, influenza virus, parainfluenza virus, respiratory syncytial virus, and Norwalk virus, also are known. For example, U.S. Pat. No. 4,767,788 discloses the use of glutaric acid to inactivate or destroy viruses. U.S. Pat. No. 4,975,217 discloses compositions containing an organic acid and an anionic surfactant, for formulation on a soap or lotion, to control viruses. U.S. Patent Publication 2002/0098159 discloses the use of a proton donating agent and a surfactant, including an antibacterial surfactant, to effect antiviral and antibacterial properties.

U.S. Pat. No. 6,034,133 discloses a virucidal hand lotion containing malic acid, citric acid, and a C₁₋₆ alcohol. U.S. Pat. No. 6,294,186 discloses combinations of a benzoic acid analog, such as salicyclic acid, and selected metal salts as being effective against viruses, including rhinovirus. U.S. Pat. No. 6,436,885 discloses a combination of known antibacterial agents with 2-pyrrolidone-5-carboxylic acid, at a pH of 2 to 5.5, to provide antibacterial and antiviral properties. U.S. Pat. No. 6,110,908 discloses a topical antiseptic containing a C₂₋₃ alcohol, a free fatty acid, and zinc pyrithione.

Organic acids in personal washing compositions also have been disclosed. For example, WO 97/46218 and WO 96/06152 disclose the use of organic acids or salts, hydrotropes, triclosan, and hydric solvents in a surfactant base for antimicrobial cleansing compositions. These publications are silent with respect to antiviral properties.

Hayden et al., Antimicrobial Agents and Chemotherapy, 26:928-929 (1984), discloses interrupting the hand-to-hand transmission of rhinovirus colds through the use of a hand lotion having residual virucidal activity. The hand lotions, containing 2% glutaric acid, were more effective than a placebo in inactivating certain types of rhinovirus. However, the publication discloses that the glutaric acid-containing lotions were not effective against a wide spectrum of rhinovirus serotypes.

A virucidal tissue designed for use by persons infected with the common cold, and including citric acid, malic acid, and sodium lauryl sulfate, is known. Hayden et al., Journal of Infectious Diseases, 152:493-497 (1985), however, reported that use of paper tissues, either treated with virus-killing substances or untreated, can interrupt the hand-to-hand transmission of viruses. Hence, no distinct advantage in preventing the spread of rhinovirus colds can be attributed to the compositions incorporated into the virucidal tissues.

An efficacious antimicrobial composition effective against both bacteria and viruses has been difficult to achieve because of the fundamental differences between a bacteria and a virus, and because of the properties of the antimicrobial agents and the effects of a surfactant on an antimicrobial agent. For example, several antimicrobial agents, like phenols, have an exceedingly low solubility in water, e.g., triclosan solubility in water is about 5 to 10 ppm (parts per million). The solubility of the antimicrobial agent is increased by adding surfactants to the composition. However, an increase in solubility of the antimicrobial agent, and, in turn, the amount of antimicrobial agent in the composition, does not necessarily lead to an increased efficacy.

Although a number of antimicrobial cleansing products currently exist, taking a variety of product forms (e.g., deodorant soaps, hard surface cleaners, and surgical disinfectants), such antimicrobial products typically incorporate high levels of alcohol and/or harsh surfactants, which can dry out and irritate skin tissues. Ideally, personal cleansing products gently cleanse the skin, cause little or no irritation, and do not leave the skin overly dry after frequent use.

Accordingly, a need exists for an antimicrobial composition that is highly efficacious against a broad spectrum of microbes, including noroviruses and Gram positive and Gram negative bacteria, in a short time period, and wherein the composition preferably can provide a persistent antiviral activity, and is mild to the skin. Compositions demonstrating improved mildness and a heightened level of viral and bacterial reduction are provided by the method and the antimicrobial compositions of the present invention.

### SUMMARY OF THE INVENTION

The present invention is directed to antimicrobial compositions that provide a rapid antibacterial effectiveness, and a rapid, and preferably persistent, effectiveness against noroviruses. The compositions provide a substantial norovirus control and a substantial reduction in Gram positive and Gram negative bacteria in less than about one minute.

More particularly, the present invention relates to antimicrobial compositions containing a disinfecting alcohol, an organic acid, an optional active antimicrobial agent, an optional gelling agent, and water, wherein the composition has a pH of about 5 or less. In preferred embodiments, the composition is capable of providing a residual layer of the organic acid on a treated surface. A present composition preferably is free of intentionally added cleansing surfactants, such as anionic, cationic, and ampholytic surfactants. The optional active antimicrobial agent can be a phenolic or a quaternary ammonium antimicrobial agent, for example.

Accordingly, one aspect of the present invention is to provide an antimicrobial composition that is highly effective at killing a broad spectrum of bacteria, including Gram positive and Gram negative bacteria such as S. aureus, S. choleraesuis, E. coli, and K. pneumoniae, while simultaneously inactivating or destroying viruses harmful to human health, particularly noroviruses.

Another aspect of the present invention is to provide a liquid, antimicrobial composition comprising:

(a) about 25% to about 95%, by weight, of a disinfecting alcohol, like a C₁₋₆ alcohol;

(b) a virucidally effective amount of an organic acid;

(c) about 0% to about 5%, by weight, of an active antimicrobial agent;

(d) 0% to about 5%, by weight, of a gelling agent, like a colloidal or a polymeric gelling agent; and

(e) water; wherein the composition has a pH of about 5 or less.

In preferred embodiments, the composition provides an essentially continuous layer or film of the organic acid on a treated surface to impart a persistent antiviral activity to the treated surface. In other preferred embodiments, the composition is free of an intentionally-added surfactant.

Another aspect of the present invention is to provide an antimicrobial composition having antibacterial and antiviral activity comprising a disinfecting alcohol, an organic acid selected from the group consisting of a monocarboxylic acid, a polycarboxylic acid, a polymeric acid having a plurality of carboxylic, phosphate, sulfonate, and/or sulfate moieties, and mixtures thereof, an optional active antimicrobial agent, and an optional gelling agent.

Another aspect of the present invention is to provide an antimicrobial composition comprising an organic acid that is substantive to the skin, and/or that fails to penetrate the skin, and/or that resists rinsing from the skin, and/or that forms an essentially continuous barrier layer on the skin. Such organic acids typically have a log P of less than one, and the compositions are effective against a broad spectrum of bacteria and exhibit a synergistic activity against noroviruses. The persistent antiviral activity is attributed, in part, to a residual layer or film of the organic acid on a treated surface, which resists removal from the skin after several rinsings, and during normal daily routines for a period of several hours.

Preferred compositions comprise one or more polycarboxylic acid, a polymeric acid, and a gelling agent. These compositions provide an effective and persistent control of noroviruses and exhibit a synergistic activity against Gram positive and Gram negative bacteria.

Another aspect of the present invention is to provide an antimicrobial composition that exhibits a substantial, and preferably persistent, control of noroviruses, and has a pH of about 2 to about 5.

Yet another aspect of the present invention is to provide an antimicrobial composition that exhibits a log reduction against Gram positive bacteria (i.e., S. aureus) of at least 2 after 30 seconds of contact.

Still another aspect of the present invention is to provide an antimicrobial composition that exhibits a log reduction against Gram negative bacteria (i.e., E. coli) of at least 2.5 after 30 seconds of contact.

Another aspect of the present invention is to provide an antimicrobial composition that exhibits a log reduction against noroviruses, such as genogroups GI, GII, and GIV, of at least 2 after 30 seconds of contact. The antimicrobial composition also preferably provides a log reduction against noroviruses of at least 2 for at least about four hours, and at least 2 for at least about six hours, after application with a 30 second contact time. In some embodiments, the antimicrobial composition provides a log reduction against noroviruses of about 2 for up to about eight hours.

Another aspect of the present invention is to provide an antimicrobial composition that resists rinsing from the skin, e.g., at least 50%, at least 60%, and preferably at least 70% of the nonvolatile components of an applied composition remains on a treated surface after three water rinsings and an effective antiviral amount of the composition remains on the skin after ten water rinsings.

Another aspect of the present invention is to provide consumer products based on an antimicrobial composition of the present invention, for example, a skin cleanser, a body splash, a surgical scrub, a wound care agent, a hand sanitizer gel, a disinfectant, a pet shampoo, a hard surface sanitizer, a lotion, an ointment, a cream, a swab, a wipe, and the like. A composition of the present invention can be a rinse-off product, but preferably is a leave-on product. The compositions are esthetically pleasing and nonirritating to the skin.

A further aspect of the present invention is to provide a method of quickly controlling noroviruses and the Gram positive and/or Gram negative bacteria populations on animal tissue, including human tissue, by contacting the tissue, like the dermis, with a composition of the present invention for a sufficient time, for example, about 15 seconds to 5 minutes or longer, e.g., about one hour, to reduce bacterial and norovirus population levels to a desired level. A further aspect of the present invention is to provide a composition that exhibits a persistent control of noroviruses on animal tissue.

Yet another aspect of the present invention is to provide a composition and method of interrupting transmission of a norovirus from animate and inanimate surfaces to an animate surface, especially human skin and mouth. Especially provided is a method and composition for controlling the transmission of norovirus by effectively controlling noroviruses present on human skin and continuing to control noroviruses for a period of about four or more hours, and up to about eight hours, after application of the composition to the skin.

These and other novel aspects and advantages of the present invention are set forth in the following, nonlimiting detailed description of the preferred embodiments.

### BRIEF DESCRIPTION OF THE FIGURES

FIGS. 1a and 1b are reflectance micrographs showing a barrier layer of nonvolatile components on a surface provided by application of a composition of the present invention to the surface;

FIGS. 1c and 1d are reflectance micrographs showing the absence of a barrier layer on a surface after application of a control composition to the surface;

FIG. 2 is a bar graph showing the log reduction against Feline Calicivirus over ten water rinses;

FIGS. 3a and 3b are bar graphs showing the log reduction against Feline Calicivirus over drying time; and

FIG. 4 contains bar graphs showing a log reduction of norovirus over time for fingerpads contacted with Composition A of Example 14 and a comparative commercial product.

### DETAILED DESCRIPTION OF THE PREFERRED EMBODIMENTS

Personal care products incorporating an active antimicrobial agent have been known for many years. Since the introduction of antimicrobial personal care products, many claims have been made that such products provide antimicrobial properties. To be most effective, an antimicrobial composition should provide a high log reduction against a broad spectrum of organisms in as short a contact time as possible. Ideally, the composition also should inactivate viruses.

As presently formulated, most commercial liquid antibacterial soap compositions provide a poor to marginal time kill efficacy, i.e., rate of killing bacteria. These compositions do not effectively control viruses.

Antimicrobial hand sanitizer compositions typically do not contain a surfactant and rely upon a high concentration of an alcohol to control bacteria. The alcohols evaporate and, therefore, cannot provide a control of noroviruses or a persistent bacterial control. The alcohols also can dry and irritate the skin.

Most current products especially lack efficacy against Gram negative bacteria, such as E. coli, which are of particular concern to human health. Compositions do exist, however, that have an exceptionally high broad spectrum antibacterial efficacy, as measured by a rapid kill of bacteria (i.e., time kill), which is to be distinguished from persistent kill. These products also lack a sufficient antiviral activity, and, in particular, an activity against noroviruses.

The present antimicrobial compositions provide excellent broad spectrum antibacterial efficacy and significantly improve antiviral efficacy against noroviruses compared to prior compositions that incorporate a high percentage of an alcohol, i.e., greater than 40%, by weight. The basis of this improved efficacy is (a) the discovery that a combination of a disinfecting alcohol and an organic acid, and especially an organic acid having a log P of less than about 1, substantially improves antiviral efficacy, and (b) the pH of a surface after application of the composition to the surface. An important aspect of the present invention is to maintain a low skin pH for an extended time to provide a persistent antiviral activity. In preferred embodiments, this is achieved by forming an essentially continuous film of the nonvolatile composition components on the skin, which provides a reservoir of the organic acids to maintain a low skin pH.

The term “essentially continuous film” means that a residue of the nonvolatile components of the composition in the form of a barrier layer is present on at least 50%, at least 60%, at least 70%, or at least 80%, preferably at least 85% or at least 90%, and more preferably at least 95%, of the area of the treated surface area. An “essentially continuous” film is demonstrated in the reflectance micrographs of the figures, which are discussed hereafter. The term “essentially continuous film” as used herein is synonymous with the term “essentially continuous layer”, “barrier layer”, and “barrier film”.

A disinfecting alcohol and an organic acid having a log P of less than one act synergistically to control noroviruses. A disinfecting alcohol and an organic acid having a log P of one or greater act synergistically to substantially improve antibacterial efficacy. A combination of a first organic acid having a log P less than one and a second organic acid having a log P of one or greater, with a disinfecting alcohol, provides a synergistic improvement in the control of noroviruses and Gram positive and Gram negative bacteria. Another basis of improved efficacy is the discovery that the antimicrobial efficacy of an active antimicrobial agent, and particularly a phenolic antimicrobial agent, can be correlated to the rate at which, and length of time, the agent has access to an active site on the microbe, and to the pH of the surface after application of the composition to the surface.

One driving force that determines the rate of active antimicrobial agent transport to the site of action is the difference in chemical potential between the site at which the agent acts and the external aqueous phase. Alternatively stated, the microbicidal activity of an active antimicrobial agent is proportional to its thermodynamic activity in the external phase. Accordingly, thermodynamic activity, as opposed to concentration, is an important variable with respect to antimicrobial efficacy. As discussed more fully hereafter, thermodynamic activity is conveniently correlated to the percent saturation of the active antimicrobial agent in the continuous aqueous phase of the composition.

Many compounds have a solubility limit in aqueous solutions termed the “saturation concentration,” which varies with temperature. Above the saturation concentration, the compound precipitates from solution. Percent saturation is the measured concentration in solution divided by the saturation concentration. The concentration of a compound in aqueous solution can be increased over the saturation concentration in water by the addition of compounds like surfactants. Surfactants not only increase the solubility of compounds in the continuous aqueous phase of the composition, but also form micelles, and can solubilize compounds in the micelles.

The % saturation of an active antimicrobial agent in any composition, including a surfactant-containing composition, ideally can be expressed as:
% saturation=[C/Cₛ]×100%,
wherein C is the concentration of antimicrobial agent in solution in the composition and Cₛ is the saturation concentration of the antimicrobial agent in the composition at room temperature. It has been theorized that the continuous aqueous phase of a surfactant-containing composition is in equilibrium with the micellar pseudophase of said composition, and further that any dissolved species, such as an antimicrobial active agent, is distributed between the aqueous continuous phase and the micellar pseudophase according to a partition law. Accordingly, the percent saturation, or alternatively the relative thermodynamic activity or relative chemical potential, of an antimicrobial active agent dissolved in a surfactant-containing composition is the same everywhere within the composition. Thus, the terms percent saturation of the antimicrobial agent “in a composition,” “in the aqueous continuous phase of a composition,” and “in the micellar pseudophase of a composition” are interchangeable.

Maximum antimicrobial efficacy is achieved when the difference in thermodynamic activities of the active antimicrobial agent between the composition and the target organism is maximized (i.e., when the composition is more “saturated” with the active ingredient). A second factor affecting antimicrobial activity is the total amount of available antimicrobial agent present in the composition, which can be thought of as the “critical dose.” It has been found that the total amount of active agent in the continuous aqueous phase of a composition greatly influences the time in which a desired level of antimicrobial efficacy is achieved, given equal thermodynamic activities. Thus, two key factors affecting the antimicrobial efficacy of an active agent in a composition are: (1) its availability, as dictated by its thermodynamic activity, i.e., percent saturation in the continuous aqueous phase of a composition, and (2) the total amount of available active agent in the solution. A third key factor is the amount of time the active antimicrobial agent is allowed to remain in contact with treated surfaces.

An ingredient in many antimicrobial cleansing compositions is a surfactant, which acts as a solubilizer, cleanser, and foaming agent. Surfactants affect the percent saturation of an antimicrobial agent in solution, or more importantly, affect the percent saturation of the active agent in the continuous aqueous phase of the composition. This effect can be explained in the case of a sparingly water-soluble antimicrobial agent in an aqueous surfactant solution, where the active agent is distributed between the aqueous (i.e., continuous) phase and the micellar pseudophase. For antimicrobial agents of exceedingly low solubility in water, such as triclosan, the distribution is shifted strongly toward the micelles (i.e., a vast majority of the triclosan molecules are present in surfactant micelles, as opposed to the aqueous phase).

The ratio of surfactant to active antimicrobial agent directly determines the amount of active antimicrobial agent present in the surfactant micelles, which in turn affects the percent saturation of the active antimicrobial agent in the continuous aqueous phase. It has been found that as the surfactant:active antimicrobial agent ratio increases, the number of micelles relative to active molecules also increases, with the micelles being proportionately less saturated with active antimicrobial agent as the ratio increases. Because an active antimicrobial agent in the continuous phase is in equilibrium with active agent in the micellar pseudophase, as the saturation of active agent in the micellar phase decreases, so does the saturation of the antimicrobial agent in the continuous phase. The converse also is true. Active antimicrobial agent solubilized in the micellar pseudophase is not immediately available to contact the microorganisms, and it is the percent saturation of active agent in the continuous aqueous phase that determines the initial antimicrobial activity of the composition. The active agent present in the surfactant micelles serves as a reservoir of active agent to replenish the continuous aqueous phase as the active agent is depleted, and helps provide a persistent antimicrobial activity.

To summarize, the thermodynamic activity, or percent saturation, of an active antimicrobial agent in the continuous aqueous phase of a composition helps drive antimicrobial activity. Further, the total amount of available active agent, and the length of time the active agent remains on the treated surface, determines the ultimate extent of efficacy. In compositions wherein the active agent is solubilized by a surfactant, the active agent present in surfactant micelles is not immediately available for antimicrobial activity. For such compositions, the percent saturation of the active agent in the composition, or alternatively the percent saturation of the active agent in the continuous aqueous phase of the composition, determines initial antimicrobial efficacy.

Although compositions having a high percent saturation of an active antimicrobial agent have demonstrated a rapid and effective antibacterial activity against Gram positive and Gram negative bacteria, control of viruses, and particularly, noroviruses, has been inadequate. Virus control on skin and inanimate surfaces is very important in controlling the transmission of numerous viral-mediated diseases.

For example, rhinoviruses are the most significant microorganisms associated with the acute respiratory illness referred to as the “common cold.” Other viruses, such as parainfluenza viruses, respiratory syncytial viruses (RSV), enteroviruses, and coronaviruses, also are known to cause symptoms of the “common cold,” but rhinoviruses are theorized to cause the greatest number of common colds. Noroviruses cause acute gastroenteritis in humans, which is the most reported viral infection after the common cold. Noroviruses are among the most difficult of the viruses to control, and have an ability to survive in 10 ppm chlorine and over a wide temperature range. Although the molecular biology of noroviruses is becoming understood, finding effective methods for preventing intestinal infections caused by noroviruses, and for preventing spread of the norovirus to noninfected subjects, has been fruitless.

It is known that gluteraldehyde, iodine, or a high chlorine concentration (i.e., greater than 1000 ppm) is an effective agent against norovirus. High phenolic concentrations, peracetic acid, and hydrogen peroxide also control noroviruses. Norovirus control using ethanol requires long contact time. None of these methods are amenable to continual use, especially on human skin, to control noroviruses. Thus, the development of compositions that deliver an immediate and persistent activity against noroviruses would be effective in reducing incidents of gastroenteritis. Likewise, a topically applied composition that exhibits antiviral activity against noroviruses would be effective in preventing and/or treating diseases caused by other caliciviruses.

Virucidal means capable of inactivating or destroying a virus. As used herein, the term “persistent antiviral efficacy” or “persistent antiviral activity” means leaving a residue or imparting a condition on animate (e.g., skin) or inanimate surfaces that provides significant antiviral activity for an extended time after application. In some embodiments, a “persistent antiviral efficacy” or “persistent antiviral activity” means leaving a barrier residue or film of antiviral agents, including organic acids, on animate (e.g., skin) or inanimate surfaces that provides significant antiviral activity for an extended time after application. The barrier residue or film can be continuous or essentially continuous, and resists removal from a treated surface during water rinsing.

A composition of the present invention preferably provides a persistent antiviral efficacy, i.e., preferably a log reduction of at least 2 within 30 seconds of contact with the composition. Antiviral activity preferably is maintained for at least about 0.5 hour, preferably at least about 1 hour, and more preferably for at least about 2 hours, at least about 3 hours, and at least about 4 hours after contact with the composition. In some preferred embodiments, antiviral activity is maintained for about six to about eight hours after contact with the composition. In some embodiments, the persistent antiviral activity is attributed, at least in part, to the reservoir of organic acids present in the barrier layer or film of the composition on a treated surface. The methodology utilized to determine persistent antiviral efficacy is discussed below.

The antimicrobial compositions of the present invention are highly effective in providing a rapid and broad spectrum control of bacteria, and a rapid and preferably persistent control of noroviruses. The highly effective compositions comprise (a) a disinfecting alcohol, (b) a virucidally effective amount of an organic acid, (c) an optional active antimicrobial agent, and (d) a gelling agent, preferably in a high percent saturation concentration, in a phase stable formulation. The compositions are surprisingly mild to the skin, and noncorrosive to inanimate surfaces. Thus, mild and effective compositions that solve the problem of bacterial and norovirus control are provided to consumers.

The disinfecting alcohol and an organic acid having a log P of less than about 1 act synergistically to control noroviruses. The disinfecting alcohol and an organic acid having a log P of 1 or greater act synergistically to control a broad spectrum of bacteria. A composition containing a first organic acid having a log P of less than one and a second organic acid having a log P of one or greater act synergistically to control noroviruses and a broad spectrum of Gram positive and Gram negative bacteria.

The antimicrobial compositions of the present invention are highly efficacious in household cleaning applications (e.g., hard surfaces, like floors, countertops, tubs, dishes, and softer cloth materials, like clothing), personal care applications (e.g., lotions, shower gels, soaps, shampoos, and wipes), and industrial, recreational, and healthcare applications (e.g., on cruise ships, in nursing homes, in food handling, and sterilization of instruments, medical devices, and gloves). The present compositions efficaciously and rapidly clean and disinfect surfaces that are infected or contaminated with Gram negative bacteria, Gram positive bacteria, and noroviruses. The present compositions preferably provide a persistent effectiveness against noroviruses.

The present compositions can be used in vitro and in vivo. In vitro means in or on nonliving things, especially on inanimate objects having hard or soft surfaces located or used where preventing viral transmission is desired, most especially on objects that are touched by human hands. In vivo means in or on animate objects, especially on mammal skin, and particularly on hands.

As illustrated in the following nonlimiting embodiments, an antimicrobial composition of the present invention comprises: (a) about 25% to about 95%, by weight, of a disinfecting alcohol; (b) a virucidally effective amount of an organic acid, and preferably a combination of organic acids; and (c) water. In preferred embodiments, the composition contains an optional gelling agent and/or an optional active antimicrobial agent. The compositions have a pH of less than about 5, and typically are capable of forming an essentially continuous film or layer of nonvolatile composition ingredients on a treated surface. The film or layer resists removal from the treated surface for several hours after application. In particular, an effective amount of composition ingredients remain on a treated surface after ten rinsings, and at least 50%, preferably at least 60%, and more preferably at least 70%, of the nonvolatile composition ingredients remains on a treated surface after three rinsings.

In embodiments wherein skin is treated, “rinsing” means gently rubbing treated skin under a moderate flow of tap water having a temperature of about 30° C. to about 40° C. for about 30 seconds, then air drying the skin. In embodiments wherein the composition comprises an active antimicrobial agent, a percent saturation of antimicrobial agent in the continuous aqueous phase preferably is at least about 50%, when measured at 25° C.

The compositions can further include an optional hydrotrope and/or polyhydric solvent, and additional optional ingredients disclosed hereafter, like pH adjusters, dyes, skin conditioners, vitamins, and perfumes. The present compositions typically are free of intentionally added surfactants, i.e., contain 0% to about 0.5%, by weight, of compounds that exhibit surface activity.

The compositions exhibit a log reduction against Gram positive bacteria of about 2 after 30 seconds contact. The compositions also exhibit a log reduction against Gram negative bacteria of about 2.5 after 30 seconds contact. The compositions further exhibit a log reduction against noroviruses, and other caliciviruses, of about 3 after 30 seconds contact, and preferably a log reduction against these viruses of at least 2.5 about five hours, and at least 2 about six to about eight hours, after contact. The compositions also are mild, and it is not necessary to rinse or wipe the compositions from the skin.

The following ingredients are present in an antimicrobial composition of the present invention.

A. Disinfecting Alcohol

An antimicrobial composition of the present invention contains about 25% to about 75%, by weight, of a disinfecting alcohol. Preferred embodiments contain about 30% to about 75%, by weight, of a disinfecting alcohol. Most preferred embodiments contain about 30% to about 70%, by weight, of a disinfecting alcohol.

As used herein, the term “disinfecting alcohol” is a water-soluble alcohol containing one to six carbon atoms, i.e., a C₁₋₆ alcohol. Disinfecting alcohols include, but are not limited to, methanol, ethanol, propanol, and isopropyl alcohol.

B. Organic Acid

A present antimicrobial composition contains an organic acid in a sufficient amount to control and inactivate noroviruses and bacteria on a surface contacted by the antimicrobial composition. The organic acid acts synergistically with the disinfecting alcohol to provide a rapid control of noroviruses and bacteria, and preferably a persistent norovirus control.

In particular, an organic acid is present in the composition in a sufficient amount such that the pH of the animate or inanimate surface contacted by the composition is lowered to degree wherein a persistent viral control is achieved. This persistent viral control is achieved regardless of whether the composition is rinsed from, or allowed to remain on, the contacted surface. The organic acid remains at least partially undissociated in the composition, and remains so when the composition is diluted, or during application and rinsing.

Upon application to a surface, such as human skin, the pH of the surface is sufficiently lowered such that a persistent viral control is achieved. In preferred embodiments, a residual amount of the organic acid remains on the skin, even after a rinsing step, preferably as a film or layer, in order to impart a persistent viral control. However, even if the organic acid is essentially completely rinsed from the surface, the surface pH has been sufficiently lowered to impart a viral control for at least 0.5 hours.

A preferred composition is a leave-on composition, i.e., is not intended to be rinsed from the skin. However, after three rinsings, at least 50% of nonvolatile composition ingredients remain on the surface, and an effective amount of the composition remains on the treated surface after ten rinsings.

Typically, an organic acid is present in a present composition in an amount of about 0.05% to about 15%, and preferably about 0.1% to about 10%, by weight of the composition. To achieve the full advantage of the present invention, the organic acid is present in an amount of about 0.15% to about 6%, by weight of the composition. In preferred embodiments, a mixture of organic acids is included in the composition. The total amount of organic acid in a composition is related to the class of organic acid used, and to the identity of the specific acid or acids used.

An organic acid included in a present antimicrobial composition preferably does not penetrate the surface to which it is applied, e.g., remains on the skin surface as opposed to penetrating the skin and forms a layer or film on the skin, together with other nonvolatile composition ingredients, e.g., an optional gelling agent and/or active antimicrobial agent. The organic acid, therefore, preferably is a hydrophobic organic acid.

In one embodiment of the present invention, the organic acid has a log P of less than one, and preferably less than 0.75. To achieve the full advantage of the present invention, the organic acid has a log P of less than 0.5. In this embodiment, the disinfecting alcohol and organic acid act synergistically to provide an effective and persistent viral control.

In another embodiment, the organic acid has a log P of 1 or greater, for example, 1 to about 100. In this embodiment, the disinfecting alcohol and organic acid effectively control nonenveloped viruses and also act synergistically to control a broad spectrum of bacteria.

It is envisioned that, by incorporating a first organic acid having a log P of less than one and a second organic acid having a log P of 1 or greater into a present composition, the first and second organic acids act synergistically with the disinfecting alcohol to provide a persistent control of noroviruses and a broad spectrum bacteria control.

As used herein, the term “log P” is defined as the log of the water-octanol partition coefficient, i.e., the log of the ratio P_{w}/Pₒ, wherein P_{w} is the concentration of an organic acid in water and Pₒ is the concentration of the organic acid in octanol, at equilibrium and 25° C. The water-octanol coefficient can be determined by the U.S. Environmental Protection Agency Procedure, “OPPTS 830.7560 Partition Coefficient (n-Octanol/Water), Generator Column Method” (1996).

Organic acids having a log P less than one typically are water insoluble, e.g., have a water solubility of less than about 0.5 wt % at 25° C. Organic acids having a log P of one or greater typically are considered water soluble, e.g., have a water solubility of at least 0.5 wt %, at 25° C.

An organic acid useful in a present antimicrobial composition comprises a monocarboxylic acid, a polycarboxylic acid, a polymeric acid having a plurality of carboxylic, phosphate, sulfonate, and/or sulfate moieties, or mixtures thereof. In addition to acid moieties, the organic acid also can contain other moieties, for example, hydroxy groups and/or amino groups. In addition, an organic acid anhydride can be used in a composition of the present invention as the organic acid. Preferred organic acids are polycarboxylic acids, polymeric carboxylic acids, and mixtures thereof.

In one embodiment, the organic acid comprises a monocarboxylic acid having a structure RCO₂H, wherein R is C₁₋₁₀alkyl, hydroxyC₁₋₃alkyl, haloC₁₋₃alkyl, phenyl, or substituted phenyl. The monocarboxylic acid preferably has a water solubility of at least about 0.05%, by weight, at 25° C. The alkyl groups can be substituted with phenyl groups and/or phenoxy groups, and these phenyl and phenoxy groups can be substituted or unsubstituted.

Nonlimiting examples of monocarboxylic acids useful in the present invention are acetic acid, propionic acid, octanoic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof. Additional substituted benzoic acids are disclosed in U.S. Pat. No. 6,294,186, incorporated herein by reference. Examples of substituted benzoic acids include, but are not limited to, salicyclic acid, 2-nitrobenzoic acid, thiosalicylic acid, 2,6-dihydroxybenzoic acid, 5-nitrosalicyclic acid, 5-bromosalicyclic acid, 5-iodosalicyclic acid, 5-fluorosalicylic acid, 3-chlorosalicylic acid, 4-chlorosalicyclic acid, 5-chlorosalicyclic acid, and mixtures thereof.

In another embodiment, the organic acid comprises a polycarboxylic acid. The polycarboxylic acid contains at least two, and up to four, carboxylic acid groups. The polycarboxylic acid also can contain hydroxy or amino groups, in addition to substituted and unsubstituted phenyl groups. Preferably, the polycarboxylic acid has a water solubility of at least about 0.05%, by weight, at 25° C.

Nonlimiting examples of polycarboxylic acids useful in the present invention include malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

Anhydrides of polycarboxylic and monocarboxylic acids also are organic acids useful in the present compositions. Preferred anhydrides are anhydrides of polycarboxylic acids. At least a portion of the anhydride is hydrolyzed to a carboxylic acid because of the pH of the composition. It is envisioned that an anhydride can be slowly hydrolyzed on a surface contacted by the composition, and thereby assist in providing a persistent antiviral activity.

In a third embodiment, the organic acid comprises a polymeric carboxylic acid, a polymeric sulfonic acid, a sulfated polymer, a polymeric phosphoric acid, and mixtures thereof. The polymeric acid has a molecular weight of about 500 g/mol to 10,000,000 g/mol, and includes homopolymers, copolymers, and mixtures thereof. The polymeric acid preferably is capable of forming a substantive film on a skin surface, and has a glass transition temperature, T_{g}, of less than about 25° C., preferably less than about 20° C., and more preferably less than about 15° C. The glass transition temperature is the temperature at which an amorphous material, such as a polymer, changes from a brittle vitreous state to a plastic state. The T_{g} of a polymer is readily determined by persons skilled in the art using standard techniques.

The polymeric acids are uncrosslinked or only very minimally crosslinked. The polymeric acids therefor are water soluble or at least water dispersible. The polymeric acids typically are prepared from ethylenically unsaturated monomers having at least one hydrophilic moiety, such as carboxyl, carboxylic acid anhydride, sulfonic acid, and sulfate.

Examples of monomers used to prepare the polymeric organic acid include, but are not limited to:

(a) Carboxyl group-containing monomers, e.g., monoethylenically unsaturated mono- or polycarboxylic acids, such as acrylic acid, methacrylic acid, maleic acid, fumaric acid, crotonic acid, sorbic acid, itaconic acid, ethacrylic acid, α-chloroacrylic acid, α-cyanoacrylic acid, β-methlacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, citraconic acid, mesaconic acid, glutaconic acid, aconitic acid, tricarboxyethylene, and cinnamic acid;

(b) Carboxylic acid anhydride group-containing monomers, e.g., monoethylenically unsaturated polycarboxylic acid anhydrides, such as maleic anhydride; and

(c) Sulfonic acid group-containing monomers, e.g., aliphatic or aromatic vinyl sulfonic acids, such as vinylsulfonic acid, allylsulfonic acid, vinyltoluenesulfonic acid, styrenesulfonic acid, sulfoethyl (meth)acrylate, 2-acrylamido-2-methylpropane sulfonic acid, sulfopropyl (meth)acrylate, and 2-hydroxy-3-(meth)acryloxy propyl sulfonic acid.

The polymeric acid can contain other copolymerizable units, i.e., other monoethylenically unsaturated comonomers, well known in the art, as long as the polymer is substantially, i.e., at least 10%, and preferably at least 25%, acid group containing monomer units. To achieve the full advantage of the present invention, the polymeric acid contains at least 50%, and more preferably, at least 75%, and up to 100%, acid group containing monomer units. The other copolymerizable units, for example, can be styrene, an alkyl acrylate, or an alkyl methacrylate.

A polymeric acid assists in forming a film or layer of residual organic acid on the skin, and assists further in forming a more continuous layer of residual organic acid on the skin. A polymeric acid typically is used in conjunction with a monocarboxylic acid and/or a polycarboxylic acid.

One preferred polymeric acid is a polyacrylic acid, either a homopolymer or a copolymer, for example, a copolymer of acrylic acid and an alkyl acrylate and/or alkyl methacrylate. Another preferred polymeric acid is a homopolymer or a copolymer of methacrylic acid.

Exemplary polymeric acids useful in the present invention include, but are not limited to:

| | (CARBOPOL 910, |
|---|---|
| | 934, 934P, 940, 941, |
| | ETD 2050; ULTREZ |
| Carbomers | 10, 21) |
| Acrylates/C20-30 Alkyl Acrylate Crosspolymer | (ULTREZ 20) |
| Acrylates/Beheneth 25 Methacrylate Copolymer | (ACULYN 28) |
| Acrylates/Steareth 20 Methacrylate Copolymer | (ACULYN 22) |
| Acrylates/Steareth 20 Methacrylate Crosspolymer | (ACULYN 88) |
| Acrylates Copolymer | (CAPIGEL 98) |
| Acrylates Copolymer | (AVALURE AC) |
| Acrylates/Palmeth 25 Acrylate Copolymer | (SYNTHALEN 2000) |
| Ammonium Acrylate Copolymers | |
| Sodium Acrylate/Vinyl Alcohol Copolymer | |
| Sodium Polymethacrylate | |
| Acrylamidopropyltrimonium Chloride/Acrylates | |
| Copolymer | |
| Acrylates/Acrylamide Copolymer | |
| Acrylates/Ammonium Methacrylate Copolymer | |
| Acrylates/C10-30 Alkyl Acrylate Crosspolymer | |
| Acrylates/Diacetoneacrylamide Copolymer | |
| Acrylates/Octylacrylamide Copolymer | |
| Acrylates/VA Copolymer | |
| Acrylic Acid/Acrylonitrogens Copolymer | |

In a preferred embodiment of the present invention, the organic acid comprises one or more polycarboxylic acid, e.g., citric acid, malic acid, tartaric acid, or a mixture of any two or all three of these acids, and a polymeric acid containing a plurality of carboxyl groups, for example, homopolymers and copolymers of acrylic acid or methacrylic acid.

C. Antimicrobial Agent

The compositions also can contain an optional active antimicrobial agent, for example, a bisguanidine (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxydiphenylether). The antimicrobial agent also can be hydrogen peroxide, benzoyl peroxide, benzyl alcohol, or a quaternary ammonium compound. An active antimicrobial composition is included in a present composition in an amount of about 0.001% to about 1%, by weight, if at all. Preferred optional antimicrobial agents are the phenolic and diphenyl compounds exemplified as follows.

An antimicrobial agent is present in a composition of the present invention in an amount of about 0.001% to about 5%, and preferably about 0.01% to about 2%, by weight of the composition. To achieve the full advantage of the present invention, the antimicrobial agent is present in an amount of about 0.05% to about 1%, by weight of the composition.

The antimicrobial compositions can be ready to use compositions, which typically contain 0.001% to about 2%, preferably 0.01% to about 1.5%, and most preferably about 0.05% to about 1%, of an antimicrobial agent, by weight of the composition. The antimicrobial compositions also can be formulated as concentrates that are diluted before use with one to about 100 parts water to provide an end use composition. The concentrated compositions typically contain greater than about 0.1% and up to about 5%, by weight, of the antimicrobial agent. Applications also are envisioned wherein the end use composition contains greater than 2%, by weight, of the active antimicrobial agent.

As discussed above, the absolute amount of antimicrobial agent present in the composition is important, as is the amount of available antimicrobial agent in the composition. The amount of available antimicrobial agent in the composition is related to the identity of the disinfecting alcohol in the composition, the amount of antimicrobial agent in the composition, and the presence and amount of a gelling agent and other optional ingredients in the composition.

To achieve the desired bacteria kill in a short contact time, like 15 to 60 seconds, the continuous aqueous phase of the composition preferably contains an amount of antimicrobial agent that is at least about 50%, preferably at least about 60%, and more preferably at least about 75%, of the saturation concentration of the antimicrobial agent in water, when measured at room temperature. To achieve the full advantage of the present invention, the continuous aqueous phase is about 95% to 100% saturated with the antimicrobial agent. The method of determining percent saturation of antibacterial agent in the composition is disclosed hereafter.

The antimicrobial agents useful in the present invention are phenolic compounds exemplified by the following classes of compounds:

(a) 2-Hydroxydiphenyl Compounds

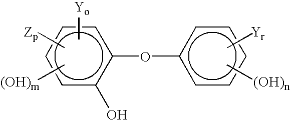

wherein Y is chlorine or bromine, Z is SO₃H, NO₂, or C₁-C₄ alkyl, r is 0 to 3, o is 0 to 3, p is 0 or l, m is 0 or 1, and n is 0 or 1.

In preferred embodiments, Y is chlorine or bromine, m is 0, n is 0 or 1, o is 1 or 2, r is 1 or 2, and p is 0.

In especially preferred embodiments, Y is chlorine, m is 0, n is 0, o is 1, r is 2, and p is 0.

A particularly useful 2-hydroxydiphenyl compound has a structure: 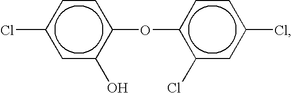
having the adopted name, triclosan, and available commercially under the tradename IRGASAN DP300, from Ciba Specialty Chemicals Corp., Greensboro, N.C. Another useful 2-hydroxydiphenyl compound is 2,2′-dihydroxy-5,5′-dibromo-diphenyl ether.

(b) Phenol Derivatives

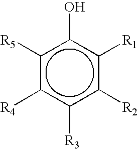

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl; and R₅ is hydro or nitro. Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsulfonic acid. Other phenol derivatives are listed in U.S. Pat. No. 6,436,885, incorporated herein by reference.

(c) Diphenyl Compounds

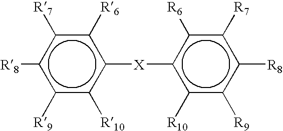

wherein X is sulfur or a methylene group, R₆ and R′₆ are hydroxy, and R₇, R′₇, R₈, R′₈, R₉, R′₉, R₁₀, and R′₁₀, independent of one another, are hydro or halo. Specific, nonlimiting examples of diphenyl compounds are hexachlorophene, tetrachlorophene, dichlorophene, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine. Other diphenyl compounds are listed in U.S. Pat. No. 6,436,885, incorporated herein by reference.

(d) Quaternary Ammonium Antibacterial Agents

Useful quaternary ammonium antibacterial agents have a general structural formula:

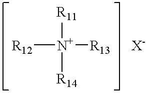

wherein at least one of R₁₁, R₁₂, R₁₃, and R₁₄ is an alkyl, aryl, or alkaryl substituent containing 6 to 26 carbon atoms. Alternatively, any two of the R substituents can be taken together, with the nitrogen atom, to form a five- or six-membered aliphatic or aromatic ring. Preferably, the entire ammonium cation portion of the antibacterial agent has a molecular weight of at least 165.

The substituents R₁₁, R₁₂, R₁₃, and R₁₄ can be straight chained or can be branched, but preferably are straight chained, and can include one or more amide, ether, or ester linkage. In particular, at least one substituent is C₆-C₂₆alkyl, C₆-C₂₆alkoxyaryl, C₆-C₂₆alkaryl, halogen-substituted C₆-C₂₆alkaryl, C₆-C₂₆alkylphenoxyalkyl, and the like. The remaining substituents on the quaternary nitrogen atom other than the above-mentioned substituent typically contain no more than 12 carbon atoms. In addition, the nitrogen atom of the quaternary ammonium antibacterial agent can be present in a ring system, either aliphatic, e.g., piperidinyl, or aromatic, e.g., pyridinyl. The anion X can be any salt-forming anion which renders the quaternary ammonium compound water soluble. Anions include, but are not limited to, a halide, for example, chloride, bromide, or iodide, methosulfate, and ethosulfate.

Preferred quaternary ammonium antibacterial agents have a structural formula:

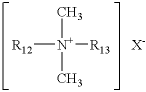

wherein R₁₂ and R₁₃, independently, are C₈-C₁₂alkyl, or R₁₂ is C₁₂-C₁₆alkyl, C₈-C₁₈alkylethoxy, or C₈-C₁₈alkylphenylethoxy, and R₁₃ is benzyl, and X is halo, methosulfate, ethosulfate, or p-toluenesulfonate. The alkyl groups R₁₂ and R₁₃ can be straight chained or branched, and preferably are linear.

The quaternary ammonium antibacterial agent in a present composition can be a single quaternary ammonium compound, or a mixture of two or more quaternary ammonium compounds. Particularly useful quaternary ammonium antibacterial agents include dialkyl(C₈-C₁₀) dimethyl ammonium chlorides (e.g., dioctyl dimethyl ammonium chloride), alkyl dimethyl benzyl ammonium chlorides (e.g., benzalkonium chloride and myristyl dimethylbenzyl ammonium chloride), alkyl methyl dodecyl benzyl ammonium chloride, methyl dodecyl xylene-bis-trimethyl ammonium chloride, benzethonium chloride, dialkyl methyl benzyl ammonium chloride, alkyl dimethyl ethyl ammonium bromide, and an alkyl tertiary amine. Polymeric quaternary ammonium compounds based on these monomeric structures also can be used in the present invention. One example of a polymeric quaternary ammonium compound is POLYQUAT®, e.g., a 2-butenyl dimethyl ammonium chloride polymer. The above quaternary ammonium compounds are available commercially under the tradenames BARDAC®, BTC®, HYAMINE®, BARQUAT®, and LONZABAC®, from suppliers such as Lonza, Inc., Fairlawn, N.J. and Stepan Co., Northfield, Ill.

Additional examples of quaternary ammonium antibacterial agents include, but are not limited to, alkyl ammonium halides, such as cetyl trimethyl ammonium bromide; alkyl aryl ammonium halides, such as octadecyl dimethyl benzyl ammonium bromide; N-alkyl pyridinium halides, such as N-cetyl pyridinium bromide; and the like. Other suitable quaternary ammonium antibacterial agents have amide, ether, or ester moieties, such as octylphenoxyethoxy ethyl dimethyl benzyl ammonium chloride, N-(laurylcocoaminoformylmethyl)pyridinium chloride, and the like. Other classes of quaternary ammonium antibacterial agents include those containing a substituted aromatic nucleus, for example, lauryloxyphenyl trimethyl ammonium chloride, cetylaminophenyl trimethyl ammonium methosulfate, dodecylphenyl trimethyl ammonium methosulfate, dodecylbenzyl trimethyl ammonium chloride, chlorinated dodecylbenzyl trimethyl ammonium chloride, and the like.

Specific quaternary ammonium antibacterial agents include, but are not limited to, behenalkonium chloride, cetalkonium chloride, cetarylalkonium bromide, cetrimonium tosylate, cetyl pyridinium chloride, lauralkonium bromide, lauralkonium chloride, lapyrium chloride, lauryl pyridinium chloride, myristalkonium chloride, olealkonium chloride, and isostearyl ethyldimonium chloride. Preferred quaternary ammonium antibacterial agents include benzalkonium chloride, benzethonium chloride, cetyl pyridinium bromide, and methylbenzethonium chloride.

(e) Anilide and Bisguanidine Antibacterial Agents

Useful anilide and bisguanadine antibacterial agents include, but are not limited to, triclocarban, carbanilide, salicylanilide, tribromosalan, tetrachlorosalicylanilide, fluorosalan, chlorhexidine gluconate, chlorhexidine hydrochloride, and mixtures thereof.

D. Gelling Agent

The present antimicrobial compositions also contain 0% to about 5%, by weight, and preferably 0.10% to about 3%, by weight, of a gelling agent. To achieve the full advantage of the present invention, the antimicrobial compositions contain about 0.25% to about 2.5%, by weight, of a gelling agent. The antimicrobial compositions typically contain a sufficient amount of gelling agent such that the composition is a viscous liquid, gel, or semisolid that can be easily applied to, and rubbed on, the skin or other surface. Persons skilled in the art are aware of the type and amount of gelling agent to include in the composition to provide the desired composition viscosity or consistency.

The term “gelling agent” as used here and hereafter refers to a compound capable of increasing the viscosity of a water-based composition, or capable of converting a water-based composition to a gel or semisolid. The gelling agent, therefore, can be organic in nature, for example, a natural gum or a synthetic polymer, or can be inorganic in nature.

As previously stated, the present compositions preferably are free of a surfactant. A surfactant often is not intentionally added to a present antimicrobial composition, but may be present in an amount of 0% to about 0.5%, by weight, because a surfactant may be present in a commercial form of a gelling agent to help disperse the gelling agent in water. A surfactant also may be present as an additive or by-product in other composition ingredients.

Surfactants preferably are omitted from the present compositions to help avoid micelle formation, which in turn solubilize the active antimicrobial compound and reduce its effectiveness. Similarly, preferred gelling agents are those that do not form micelles, and do not complex or bind with the active antimicrobial agents, or otherwise adversely effect the antimicrobial properties of the antimicrobial agent. In preferred embodiments, the identity and amount of gelling agent and other composition ingredients are selected such that the active antimicrobial agent, if present at all, is present in an amount of at least 50% of saturation, when measured at 25° C.

The following are nonlimiting examples of gelling agents that can be used in the present invention. In particular, the following compounds, both organic and inorganic, act primarily by thickening or gelling the aqueous portion of the composition:

acacia, agar, algin, alginic acid, ammonium alginate, ammonium chloride, ammonium sulfate, amylopectin, attapulgite, bentonite, C₉₋₁₅ alcohols, calcium acetate, calcium alginate, calcium carrageenan, calcium chloride, caprylic alcohol, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, fruit pectin, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-2M, PEG-5M, polyvinyl alcohol, potassium alginate, potassium carrageenan, potassium chloride, potassium sulfate, potato starch, propylene glycol alginate, sodium carboxymethyl dextran, sodium carrageenan, sodium cellulose sulfate, sodium chloride, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, polyvinylpyrrolidone and derivatives thereof, vinyl ether derivatives (methyl vinyl ether, ethyl vinyl ether, butyl vinyl ether, isobutyl vinyl ether, polymethyl vinyl ether/maleic acid), quaternized vinylpyrrolidone/quaternized dimethylamino ethyl pyrrolidone-based polymers and methacrylate copolymers, vinylcaprolactam/vinylpyrrolidone dimethylamino ethylmethacrylate polymers, vinylpyrrolidone/dimethyl amino ethylmethacrylate copolymers, acid stable and naturally occurring derivatives of guar and modified guar, modified or substituted xanthan, and carboxypropyl cellulose, and mixtures thereof.

The following additional nonlimiting examples of gelling agents act primarily by thickening the nonaqueous portion of the composition:

abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, triisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

Exemplary gelling agents useful in the present invention include, but are not limited to,

| | |
|---|---|
| Polyethylene Glycol & Propylene Glycol & Water | (ACULYN 44) |
| Ammonium Acrylatedimethyltaurate/VP Copolymer | (ARISTOFLEX AVC) |
| Glyceryl Stearate & PEG 100 Stearate | (ARLACEL 165) |
| Polyethylene(2)Stearyl Ether | (BRIJ 72) |
| Polyoxyethylene(21)Stearyl Ether | (BRIJ 721) |
| Silica | (CAB-O-SIL) |
| Polyquaternium 10 | (CELQUAT CS230M) |
| Cetyl Alcohol | |
| Cetearyl Alcohol & Cetereth 20 | (COSMOWAX P) |
| Cetearyl Alcohol & Dicetyl Phosphate & Ceteth-10 | (CRODAFOS CES) |
| Phosphate | |
| Ceteth-20 Phosphate & Cetearyl Alcohol & Dicetyl | (CRODAFOS CS-20 Acid) |
| Phosphate | |
| Cetearyl Alcohol & Cetereth 20 | (EMULGADE NI 1000) |
| Sodium Magnesium Silicate | (LAPONITE XLG) |
| Cetyl Alcohol & Stearyl Alcohol & Stearalkonium | (MACKADET CBC) |
| Chloride & Dimethyl Stearamine & Lactic Acid | |
| Cetearyl Alcohol & Stearamidopropyldimethylamine & | (MACKERNIUM Essential) |
| Stearamidopropylalkonium Chloride | |
| Stearalkonium Chloride | (MACKERNIUM SDC-85) |
| Cetearyl Alcohol & Stearamidopropyldimethylamine & | (MACKERNIUM Ultra) |
| Stearamidopropylalkonium Chloride & Silicone | |
| Quaternium 16 | |
| Cetearyl Alcohol & Cetearyl Glucoside | (MONTANOV 68EC) |
| Hydroxyethylcellulose | (NATROSOL 250 HHR CS) |
| Polyquaternium-37 & Mineral Oil & Trideceth-6 | (SALCARE SC 95) |
| Polyquaternium-32 & Mineral Oil & Trideceth-6 | (SALCARE SC 96) |
| Stearic Acid | |
| Cetyl Hydroxyethylcellulose | (NATROSOL Plus 330 CS) |
| Polyvinyl Alcohol, PVP-K30, Propylene Glycol | |
| Stearic Acid, Behenyl Alcohol, Glyceryl Stearate, | (PROLIPID 141) |
| Lecithin, C12-16 Alcohols, Palmic Acid | |
| Beeswax | (saponified beeswax) |
| Beeswax | (synthetic beeswax) |
| Water, Beeswax, Sesame Oil, Lecithin, Methyl paraben | (beesmilk) |
| Polyquaternium 10 | (CELQUAT SC240C) |
| Sodium Acrylate/Sodium Acrylodimethyl Taurate | (SIMULGEL EG) |
| Copolymer & Isohexadecane & Polysorbate 80 | |
| Polyquaternium 44 | (LUVIQUAT Care) |

E. Carrier

The carrier of the present antimicrobial composition comprises water.

F. Optional Ingredients

An antimicrobial composition of the present invention also can contain optional ingredients well known to persons skilled in the art. The particular optional ingredients and amounts that can be present in the composition are discussed hereafter.

The optional ingredients are present in a sufficient amount to perform their intended function and not adversely affect the antimicrobial efficacy of the composition, and in particular not adversely affect the synergistic effect provided by the disinfecting alcohol and organic acid, or a layer or film formed on a treated surface by the nonvolatile components of the composition. Optional ingredients typically are present, individually and collectively, from 0% to about 50%, by weight of the composition.

Classes of optional ingredients include, but are not limited to, hydrotropes, polyhydric solvents, dyes, fragrances, pH adjusters, thickeners, viscosity modifiers, chelating agents, skin conditioners, emollients, preservatives, vitamins, buffering agents, foam stabilizers, antioxidants, foam enhancers, chelating agents, opacifiers, and similar classes of optional ingredients known to persons skilled in the art.

A hydrotrope, if present at all, is present in an amount of about 0.1% to about 30%, and preferably about 1% to about 20%, by weight of the composition. To achieve the full advantage of the present invention, a composition can contain about 2% to about 15%, by weight, of a hydrotrope.

A hydrotrope is a compound that has an ability to enhance the water solubility of other compounds. A hydrotrope utilized in the present invention lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A polyhydric solvent, if present at all, is present in an amount of about 0.1% to about 50%, and preferably about 5% to about 40%, by weight of the composition. To achieve the full advantage of the present invention, the polyhydric solvent is present in an amount of about 10% to about 30% by weight of the composition. In contrast to a disinfecting alcohol, a polyhydric solvent contributes minimally, if at all, to the antimicrobial efficacy of the present composition.

A “polyhydric solvent” is a water-soluble organic compound containing two to six, and typically two or three, hydroxyl groups. The term “water-soluble” means that the polyhydric solvent has a water solubility of at least 0.1 g of polyhydric solvent per 100 g of water at 25° C. There is no upper limit to the water solubility of the polyhydric solvent, e.g., the polyhydric solvent and water can be soluble in all proportions.

The term “polyhydric solvent” therefore encompasses water-soluble diols, triols, and polyols. Specific examples of hydric solvents include, but are not limited to, ethylene glycol, propylene glycol, glycerol, diethylene glycol, dipropylene glycol, tripropylene glycol, hexylene glycol, butylene glycol, 1,2,6-hexanetriol, sorbitol, PEG-4, and similar polyhydroxy compounds.

Specific classes of optional ingredients include inorganic phosphates, sulfates, and carbonates as buffering agents; EDTA and phosphates as chelating agents; and acids and bases as pH adjusters.

Examples of preferred classes of optional basic pH adjusters are ammonia; mono-, di-, and tri-alkyl amines; mono-, di-, and tri-alkanolamines; alkali metal and alkaline earth metal hydroxides; and mixtures thereof. However, the identity of the basic pH adjuster is not limited, and any basic pH adjuster known in the art can be used. Specific, nonlimiting examples of basic pH adjusters are ammonia; sodium, potassium, and lithium hydroxide; monoethanolamine; triethylamine; isopropanolamine; diethanolamine; and triethanolamine.

Examples of preferred classes of optional acidic pH adjusters are the mineral acids. Nonlimiting examples of mineral acids are hydrochloric acid, nitric acid, phosphoric acid, and sulfuric acid. The identity of the acidic pH adjuster is not limited and any acidic pH adjuster known in the art, alone or in combination, can be used.

The composition also can contain a cosolvent or a clarifying agent, such as a polyethylene glycol having a molecular weight of up to about 4000, methylpropylene glycol, an oxygenated solvent of ethylene, propylene, or butylene, or mixtures thereof. The cosolvent or clarifying agent can be included as needed to impart stability and/or clarity to the composition and may be present in the residual film or layer of the composition on a treated surface.

A surfactant can be included in a composition in an amount of 0% to about 15%, and typically 0.1% to about 10%, by weight, of the composition. More typically, if present at all, the composition contains about 0.2% to about 7%, by weight of the surfactant. The optional surfactant is stable at the pH of the composition and is compatible with the other ingredients present in the composition.

The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

The compositions, therefore, can contain an anionic surfactant having a hydrophobic moiety, such as a carbon chain including about 8 to about 30 carbon atoms, and particularly about 12 to about 20 carbon atoms, and further has a hydrophilic moiety, such as sulfate, sulfonate, carbonate, phosphate, or carboxylate. Often, the hydrophobic carbon chain is etherified, such as with ethylene oxide or propylene oxide, to impart a particular physical property, such as increased water solubility or reduced surface tension to the anionic surfactant.

Suitable anionic surfactants include, but are not limited to, compounds in the classes known as alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylaryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates, acyl glutamates, alkyl sulfoacetates, acylated peptides, acyl lactylates, anionic fluoro surfactants, and mixtures thereof. Additional anionic surfactants are listed in McCutcheon's Emulsifiers and Detergents, 1993 Annuals, (hereafter McCutcheon's), McCutcheon Division, MC Publishing Co., Glen Rock, N.J., pp. 263-266, incorporated herein by reference. Numerous other anionic surfactants, and classes of anionic surfactants, are disclosed in U.S. Pat. No. 3,929,678 and U.S. Patent Publication No. 2002/0098159, each incorporated herein by reference.

Specific, nonlimiting classes of anionic surfactants useful in the present invention include, but are not limited to, a C₈-C₁₈ alkyl sulfonate, a C₈-C₁₈ alkyl sulfate, a C₈-C₁₈ fatty acid salt, a C₈-C₁₈ alkyl ether sulfate having one or two moles of ethoxylation, a C₈-C₁₈ alkamine oxide, a C₈-C₁₈ alkoyl sarcosinate, a C₈-C₁₈ sulfoacetate, a C₈-C₁₈ sulfosuccinate, a C₈-C₁₈ alkyl diphenyl oxide disulfonate, a C₈-C₁₈ alkyl carbonate, a C₈-C₁₈ alpha-olefin sulfonate, a methyl ester sulfonate, and mixtures thereof. The C₈-C₁₈ alkyl group contains eight to eighteen carbon atoms, and can be straight chain (e.g., lauryl) or branched (e.g., 2-ethylhexyl). The cation of the anionic surfactant can be an alkali metal (preferably sodium or potassium), ammonium, C₁-C₄ alkylammonium (mono-, di-, tri-), or C₁-C₃ alkanolammonium (mono-, di-, tri-). Lithium and alkaline earth cations (e.g., magnesium) can be used, but are not preferred.

Specific surfactants include, but are not limited to, lauryl sulfates, octyl sulfates, 2-ethylhexyl sulfates, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, ricinoleates, cetyl sulfates, and similar surfactants. Additional examples of surfactants can be found in “CTFA Cosmetic Ingredient Handbook,” J. M. Nikitakis, ed., The Cosmetic, Toiletry and Fragrance Association, Inc., Washington, D.C. (1988) (hereafter CTFA Handbook), pages 10-13, 42-46, and 87-94, incorporated herein by reference.

The compositions also can contain nonionic surfactants. Typically, a nonionic surfactant has a hydrophobic base, such as a long chain alkyl group or an alkylated aryl group, and a hydrophilic chain comprising a sufficient number (i.e., 1 to about 30) of ethoxy and/or propoxy moieties. Examples of classes of nonionic surfactants include ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, and mixtures thereof.

Exemplary nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof.

Numerous other nonionic surfactants are disclosed in McCutcheon's, at pages 1-246 and 266-272; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Handbook, at pages 86-94, each incorporated herein by reference.

In addition to anionic and nonionic surfactants, cationic, ampholytic, and amphoteric surfactants can be used in the compositions. Useful cationic surfactants include those having a structural formula

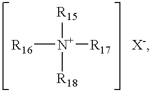

wherein R₁₅ is an alkyl group having about 12 to about 30 carbon atoms, or an aromatic, aryl, or alkaryl group having about 12 to about 30 carbon atoms; R₁₆, R₁₇, and R₁₈, independently, are selected from the group consisting of hydrogen, an alkyl group having 1 to about 22 carbon atoms, or aromatic, aryl, or alkaryl groups having from about 12 to about 22 carbon atoms; and X is a compatible anion, preferably selected from the group consisting of chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁₅, R₁₆, R₁₇, and R₁₈ also can contain ester and/or ether linkages, or hydroxy or amino group substituents (e.g., the alkyl groups can contain polyethylene glycol and polypropylene glycol moieties).

Preferably, R₁₅ is an alkyl group having about 12 to about 22 carbon atoms; R₁₆ is H or an alkyl group having 1 to about 22 carbon atoms; and R₁₇ and R₁₈, independently are H or an alkyl group having 1 to about 3 carbon atoms. More preferably, R₁₅ is an alkyl group having about 12 to about 22 carbon atoms, and R₁₆, R₁₇, and R₁₈ are H or an alkyl group having 1 to about 3 carbon atoms.

Other useful cationic surfactants include amino-amides, wherein in the above structure R₁₀ alternatively is R₁₉CONH—(CH₂)ₙ, wherein R₁₉ is an alkyl group having about 12 to about 22 carbon atoms, and n is an integer of 2 to 6, more preferably 2 to 4, and most preferably 2 to 3. Nonlimiting examples of these cationic surfactants include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof.

Additional quaternary ammonium salts include those wherein the C₁₂-C₃₀ alkyl carbon chain is derived from a tallow fatty acid or from a coconut fatty acid. The term “tallow” refers to an alkyl group derived from tallow fatty acids (usually hydrogenated tallow fatty acids), which generally has mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, and mixtures thereof. An example of a quaternary ammonium compound having an alkyl group with an ester linkage is ditallowyl oxyethyl dimethyl ammonium chloride.

Ampholytic surfactants, i.e., amphoteric and zwitterionic surfactants, can be broadly described as derivatives of secondary and tertiary amines having straight chain or branched aliphatic radicals, and wherein one of the aliphatic substituents contains from about 8 to about 18 carbon atoms and at least one of the aliphatic substituents contains an anionic water-solubilizing group, e.g., carboxy, sulfonate, or sulfate.

More particularly, one class of ampholytic surfactants include sarcosinates and taurates having the general structural formula 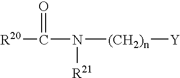
wherein R²⁰ is C₁₁-C₂₁, alkyl, R²¹ is hydrogen or C₁-C₂ alkyl, Y is CO₂M or SO₃M, M is an alkali metal, and n is a number 1 through 3.

Another class of ampholytic surfactants is the amide sulfosuccinates having the structural formula

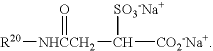

The following classes of ampholytic surfactants also can be used: 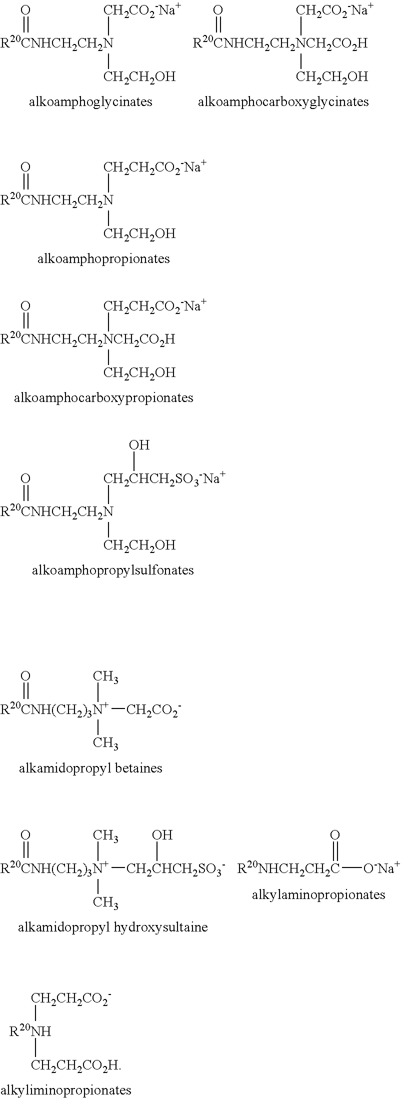
Additional classes of ampholytic surfactants include the phosphobetaines and the phosphitaines.

Specific, nonlimiting examples of ampholytic surfactants useful in the present invention are sodium coconut N-methyl taurate, sodium oleyl N-methyl taurate, sodium tall oil acid N-methyl taurate, sodium palmitoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamido-bis-(2-hydroxyethyl)propylsultaine, disodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, capryloamphocarboxypropionate, dihydroxyethyl tallow glycinate, cocamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, lauric myristic amido propyl monosodium phosphitaine, and mixtures thereof.

Useful amphoteric surfactants also include the amine oxides. Amine oxides have a general structural formula wherein the hydrophilic portion contains a nitrogen atom that is bound to an oxygen atom with a semipolar bond.

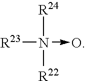

R²², R²³, and R²⁴ can be a saturated or unsaturated, branched, or unbranched alkyl or alkenyl group having 1 to about 24 carbon atoms. Preferred amine oxides contain at least one R group that is an alkyl chain of 8 to 22 carbon atoms. Nonlimiting examples of amine oxides include alkyl dimethyl amine oxides, such as decylamine oxide, cocamine oxide, myristamine oxide, and palmitamine oxide. Also useful are the alkylaminopropylamine oxides, for example, coamidopropylamine oxide and stearamidopropylamine oxide.

Nonlimiting examples of preferred surfactants utilized in a composition include those selected from the group consisting of alkyl sulfates; alkyl ether sulfates; alkyl benzene sulfonates; alpha olefin sulfonates; primary or secondary alkyl sulfonates; alkyl phosphates; acyl taurates; alkyl sulfosuccinates; alkyl sulfoacetates; sulfonated fatty acids; alkyl trimethyl ammonium chlorides and bromides; dialkyl dimethyl ammonium chlorides and bromides; alkyl dimethyl amine oxides; alkylamidopropyl amine oxides; alkyl betaines; alkyl amidopropyl betaines; and mixtures thereof. More preferred surfactants include those selected from the group consisting of alkyl sulfates; alkyl ether sulfates; alkyl benzene sulfonates; alpha olefin sulfonates; primary or secondary alkyl sulfonates; alkyl dimethyl amine oxides; alkyl betaines; and mixtures thereof.

An optional alkanolamide to provide composition thickening can be, but is not limited to, cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. Alkanolamides are noncleansing surfactants and are added, if at all, in small amounts to thicken the composition.

G. pH

The pH of a present antimicrobial composition is about 5 or less, and preferably about 4.5 or less, at 25° C. To achieve the full advantage of the present invention, the pH is less than about 4. Typically, the pH of a present composition is about 2 to less than about 5, and preferably about 2.5 to about 4.5.

The pH of the composition is sufficiently low such that at least a portion of the organic acid is in the protonated form. The organic acid then has the capability of lowering surface pH, including skin pH, to provide an effective norovirus control, without irritating the surface. The organic acid also may deposit on the surface to form a layer or film, and resist removal by rinsing, to provide a persistent antiviral effect.

To demonstrate the new and unexpected results provided by the antimicrobial compositions of the present invention, the following examples are prepared, and the ability of the compositions to control Gram positive and Gram negative bacteria, and to control norovirus, is determined. The weight percentage listed in the examples represents the actual, or active, weight amount of each ingredient present in the composition. The compositions are prepared by blending the ingredients, as understood by those skilled in the art and as described below.

The following methods are used in the preparation and testing of the examples:

(a) Determination of Rapid Germicidal (Time Kill) Activity of Antibacterial Products. The activity of antibacterial compositions is measured by the time kill method, whereby the survival of challenged organisms exposed to an antibacterial test composition is determined as a function of time. In this test, a diluted aliquot of the composition is brought into contact with a known population of test bacteria for a specified time period at a specified temperature. The test composition is neutralized at the end of the time period, which arrests the antibacterial activity of the composition. The percent or, alternatively, log reduction from the original bacteria population is calculated.

In general, the time kill method is known to those skilled in the art.

The composition can be tested at any concentration up to 100%. The choice of which concentration to use is at the discretion of the investigator, and suitable concentrations are readily determined by those skilled in the art. For example, viscous samples usually are tested at 50% dilution, whereas nonviscous samples are not diluted. The test sample is placed in a sterile 250 ml beaker equipped with a magnetic stirring bar and the sample volume is brought to 100 ml, if needed, with sterile deionized water. All testing is performed in triplicate, the results are combined, and the average log reduction is reported.

The choice of contact time period also is at the discretion of the investigator. Any contact time period can be chosen. Typical contact times range from 15 seconds to 5 minutes, with 30 seconds and 1 minute being typical contact times. The contact temperature also can be any temperature, typically room temperature, or about 25° C.

The bacterial suspension, or test inoculum, is prepared by growing a bacterial culture on any appropriate solid media (e.g., agar). The bacterial population then is washed from the agar with sterile physiological saline and the population of the bacterial suspension is adjusted to about 10⁸ colony forming units per ml (cfu/ml).

The table below lists the test bacterial cultures used in the tests and includes the name of the bacteria, the ATCC (American Type Culture Collection) identification number, and the abbreviation for the name of the organism used hereafter. S. aureus is a Gram positive bacteria, whereas E. coli, K. pneum, and S. choler. are Gram negative bacteria.

| Organism Name | ATCC # | Abbreviation |
|---|---|---|
| Staphylococcus aureus | 6538 | S. aureus |
| Escherichia coli | 11229 | E. coli |
| Klebsiella pneumoniae | 10031 | K. pneum. |
| Salmonella choleraesuis | 10708 | S. choler. |

The beaker containing the test composition is placed in a water bath (if constant temperature is desired), or placed on a magnetic stirrer (if ambient laboratory temperature is desired). The sample then is inoculated with 1.0 ml of the test bacteria suspension. The inoculum is stirred with the test composition for the predetermined contact time. When the contact time expires, 1.0 ml of the test composition/bacteria mixture is transferred into 9.0 ml of Neutralizer Solution. Decimal dilutions to a countable range then are made. The dilutions can differ for different organisms. Selected dilutions are plated in triplicate on TSA+ plates (TSA+ is Trypticase Soy Agar with Lecithin and Polysorbate 80). The plates then are incubated for 24±2 hours, and the colonies are counted for the number of survivors and the calculation of percent or log reduction. The control count (numbers control) is determined by conducting the procedure as described above with the exception that deionized water is used in place of the test composition. The plate counts are converted to cfu/ml for the numbers control and samples, respectively, by standard microbiological methods.

The log reduction is calculated using the formula

$$Log reduction = {log}_{10} ( numbers controlled ) - \\ {log}_{10} ( test sample survivors ) .$$

The following table correlates percent reduction in bacteria population to log reduction:

| % Reduction | Log Reduction |
|---|---|
| 90 | 1 |
| 99 | 2 |
| 99.9 | 3 |
| 99.99 | 4 |
| 99.999 | 5 |

(b) Antiviral Residual Efficacy Test

References: S. A. Sattar, Standard Test Method for Determining the Virus-Eliminating Effectiveness of Liquid Hygienic Handwash Agents Using the Fingerpads of Adult Volunteers, Annual Book of ASTM Standards. Designation E1838-96, incorporated herein by reference in its entirety, and referred to as “Sattar I”; and S. A. Sattar et al., Chemical Disinfection to Interrupt Transfer of Rhinovirus Type 14 from Environmental Surfaces to Hands, Applied and Environmental Microbiology, Vol. 59, No. 5, May, 1993, pp. 1579-1585, incorporated herein by reference in its entirety, and referred to as “Sattar II.”

The method used to determine the Antiviral Index of the present invention is a modification of that described in Sattar I, a test for the virucidal activity of liquid hand washes (rinse-off products). The method is modified in this case to provide reliable data for leave-on products.

Modifications of Sattar I include the product being delivered directly to the skin as described below, virus inoculation of the fingerpads as described below, and viral recovery using ten-cycle washing. The inoculated skin site then is completely decontaminated by treating the area with 70% dilution of ethanol in water.

Procedure:

Ten-minute Test:

The hands then are treated with 70% ethanol and air dried.

Test product (typically 1.0 ml up to 5.0 mL) is applied to the hands, except for the thumbs, and allowed to dry.

About 10 minutes (±30 seconds) after product application, 10 μl of a Feline Calicivirus (FVC), the accepted surrogate for norovirus (ATCC VR-782, approximately 1×10⁶ TCID₅₀/0.1 mL (tissue culture infectious dose)/ml), is topically applied using a micropipette to various sites on the hand within a designated skin surface area known as fingerpads. At this time, a solution of Feline Calicivirus also is applied to the untreated thumb in a similar manner.

After a dry-down period of 7-10 minutes, the virus then is eluted from each of the various skin sites with 1 ml of eluent (Minimal Essential media (MEM)+1% pen-strep-glutamate), washing 10 times per site.

The inoculated skin site then is completely decontaminated by treating the area with a 1:10 dilution of domestic bleach (CLOROX® 5.25% sodium hypochlorite) in tap water, then rinsing with 70% ethanol. Viral titers are determined using standard techniques, i.e., plaque assays or TCID₅₀ (Tissue Culture Infectious Dose).

One-hour test:

Subjects are allowed to resume normal activities (with the exception of washing their hands) between the 1-hour and 3-hour timepoints. After one hour, a Feline Calicivirus suspension is applied to and eluted from designated sites on the fingerpads exactly as described in above for the 10-minute test.

Examples 1-11 demonstrate the ability of a present composition to control viruses and bacteria, and to form a barrier layer on a treated surface. Examples 12-21 demonstrate an ability of the composition to control noroviruses. Examples 22-25 illustrate additional nonlimiting examples of the present antimicrobial compositions.

### EXAMPLE 1

The following compositions were prepared.

| Sample | Composition (by wt %) |
|---|---|
| A | 62% ethanol in water |
| B | 30% ethanol in water |
| C | 2% salicylic acid in 62% ethanol/water |
| D | 2% salicylic acid in 30% ethanol/water |
| E | 2% salicylic acid in dipropylene glycol/water |

The samples were tested for antiviral activity against Rhinovirus 1A and Rotavirus Wa in a time kill suspension test. The following table summarizes the results of the test.

| | Log 10 Reduction of Virus | | | |
|---|---|---|---|---|
| | Rhinovirus 1A | | Rotavirus Wa | |
| Sample | 30 sec | 1 min | 30 sec | 1 min |
| A | <1 log | <1 log | <1 log | <1 log |
| B | <1 log | <1 log | <1 log | <1 log |

| | | |
|---|---|---|
| C | Complete elimination | Complete elimination |
| D | Complete elimination | Complete elimination |
| E | Incomplete inactivation | Incomplete inactivation |

This example illustrates the synergistic antiviral effect provided by the combination of a disinfecting alcohol and an organic acid having a log P of less than one. Samples A and B show that a disinfecting alcohol alone does not provide an acceptable control of viruses. Sample E shows that salicylic acid dissolved in dipropylene glycol and water does not completely inactivate the tested virus serotypes. However, Samples C and D, which are compositions of the present invention, completely eliminate the tested virus serotypes.

### EXAMPLE 2

The following antiviral composition, which is capable of reducing skin pH, was prepared and applied to the fingerpads of human volunteers:

Sample 2

| Material | Percent (by weight) |
|---|---|
| Ethanol | 70.0 |
| Deionized water | 19.8 |
| ULTREZ ® 20¹⁾ | 1.0 |
| Isopropyl Palmitate | 1.0 |
| Mineral oil | 1.0 |
| DC 200 silicone fluid | 1.0 |
| Cetyl alcohol | 1.0 |
| Citric acid | 2.0 |
| Malic acid | 2.0 |
| GERMABEN II²⁾ | 1.0 |
| Triethanolamine | 0.05 |
| | 100.0 |

¹⁾Acrylate/C10-30 Alkyl Acrylate Crosspolymer;
²⁾Preservative containing propylene glycol, diazolidinyl urea, methylparaben, and propylparaben.

The pH of Sample 2 was 3.1.

In the test, Sample 2 was applied to the fingerpads of all fingers, except the thumbs, of eight volunteers. The thumbs were control sites. The volunteers were divided into fours groups of two each. Each group I-IV then was challenged at a predetermined time with rhinovirus titer on all the fingerpads of each hand to determine the time-dependent efficacy of the test composition. At the time appropriate for each group, the skin pH of the fingerpads also was measured to determine the time course of skin pH in response to the test composition. The predetermined test time for rhinoviral challenge and skin pH measurement for each group I-IV were 5 minutes, 1 hour, 2 hours, and 4 hours, respectively. The following table summarizes the average log (rhinoviral titer inoculum), average skin pH, and average log (rhinoviral titer recovered) from the test fingerpads of the volunteers in the study, organized by group.

| | Initial skin | | | |
|---|---|---|---|---|
| | pH after | | | Log |
| | application | Skin pH at test | Log [Inoculum | [Recovered |
| Group | (average) | time (average) | Titer] (average) | Titer] (average) |
| I | 3.0 | 3.0 | 3.9 | 0.23 |
| II | 2.8 | 3.4 | 4.0 | 0.23 |
| III | 3.0 | 3.8 | 3.8 | 0.23 |
| IV | 3.0 | 3.8 | 4.3 | 0.23 |

The data for each group (i.e., different time points) shows that the average recovered rhinoviral titer is less than 1 virus particle, or below the detection limit of the test. This data illustrates the efficacy of the present method after 4 hours and further demonstrates that a skin pH of less than about 4 is effective at completely eliminating a virus challenge. The combination of citric acid, malic acid, and polymeric acid (i.e., ULTREZ® 20) provided a residual barrier layer of organic acids on the fingerpads, which enhanced the persistent antiviral activity of the composition.

### EXAMPLE 3

The clean fingerpads of test subjects were treated with the following compositions. Baseline skin pH readings were measured from the fingerpads prior to treatment with the compositions. Skin pH measurements also were taken immediately after the composition dried on the fingerpads, then again after four hours.

| | | | Average | | | % |
|---|---|---|---|---|---|---|
| | | Average | Skin | Viral | | Hands |
| | | Skin pH | pH (T = | Log 10 | | with |
| Sample | Composition (by wt %) | (T = 0) | 4 hr) | Reduction | | Virus |
| A | 2% citric acid, 2% malic | 2.81 | 3.23 | >3 | log₁₀ | 0 |
| | acid, 62% ETOH, 1.25% | | | | | |
| | hydroxyethylcellulose | | | | | |
| B | 2% citric acid, | 2.64 | 3.03 | >3 | log₁₀ | 0 |
| | 2% tartaric acid, | | | | | |
| | 62% ETOH, 1.25% | | | | | |
| | hydroxyethylcellulose | | | | | |
| C | 2% malic acid, 2% | 2.66 | 2.94 | >3 | log₁₀ | 0 |
| | tartaric acid, 62% | | | | | |
| | ETOH, 1.25% | | | | | |
| | hydroxyethylcellulose | | | | | |
| D | 62% ETOH, 1.25% | 5.53 | 5.13 | <0.5 | log₁₋ | 100 |
| | hydroxyethylcellulose | | | | | |
| E | 2% citric acid, 2% malic | 2.90 | 3.72 | >3 | log₁₀ | 0 |
| | acid, 70% ETOH, 1% | | | | | |
| | polyacrylic acid | | | | | |
| F | 70% ETOH, 1% | 4.80 | 5.16 | 2.0 | log₁₀ | 100 |
| | polyacrylic acid | | | | | |
| G | 70% ETOH, 1.25% | 5.3 | 5.25 | <0.5 | log₁₀ | 100 |
| | hydroxyethylcellulose | | | | | |

¹⁾ETOH is ethanol

Four hours after treatment of the fingerpads with Samples A-G, Rhinovirus 39 at a titer of 1.3×10³ pfu (plaque forming units) was applied to fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 39, i.e., a greater than 3 log reduction, was achieved using the acid-containing compositions containing a mixture of two of citric acid, malic acid, and tartaric acid. The presence of hydroxyethylcellulose or polyacrylic acid assisted in forming a more continuous film or layer of organic acids on the treated fingerpads, which in turn enhanced the persistent antiviral activity of the compositions.

### EXAMPLE 4 ANTIBACTERIAL ACTIVITY

Log Reduction

| | S. aureus | | E. coli | |
|---|---|---|---|---|
| | ATCC 6538 | | ATCC 11229 | |
| Sample | 30 seconds¹⁰ | 60 seconds¹⁾ | 30 seconds | 60 seconds |
| A | >4.91 | >4.91 | >5.00 | >5.00 |
| B | >4.91 | >4.91 | >5.00 | >5.00 |

¹⁾Contact time on the skin
A. 62% Ethanol, 2% citric acid, 2% malic acid, 1.25% hydroxyethylcellulose
B. 62% Ethanol, 2% citric acid, 2% malic acid, 1.25% hydroxyethylcellulose, and skin emollients

This example illustrates that compositions of the present invention also provide a rapid and broad spectrum antibacterial activity.

### EXAMPLE 5

The clean fingerpads of test subjects were treated with the following composition. Baseline skin pH readings were measured from the fingerpads prior to treatment with the compositions. Skin pH measurements also were taken immediately after the composition dried on the fingerpads.

Immediately after treatment of the fingerpads with the composition, Rhinovirus 14 at a titer of 1.4×10⁴ pfu (plaque forming units) was applied to the fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 14 was achieved with the acid-containing composition resulting in a 4 log reduction.

| | | | Viral Log 10 | |
|---|---|---|---|---|
| | | Solution | Reduction 30 | % Hands |
| Sample | Composition (by wt %) | pH | seconds | with Virus |
| A | 2% citric acid, 2% malic | 3.10 | 4 log | 0 |
| | acid, 70% ETOH, 1% | | | |
| | polyacrylic acid | | | |

### EXAMPLE 6

The following compositions were prepared to test the effect of organic acids and organic acid blends on skin pH and antiviral efficacy.

| | | Average | Average | Viral |
|---|---|---|---|---|
| | | Skin pH | Skin pH | Log10 |
| Sample | Composition (by wt %) | (T = 0) | (T = 2 hr) | Reduction |
| A | 4% citric acid in 70% | 2.97 | 3.64 | >3 log₁₀ |
| | ethanol/water | | | |
| B | 4% malic acid in 70% | 2.91 | 3.94 | >3 log₁₀ |
| | ethanol/water | | | |
| C | 2% citric acid and 2% malic | 2.99 | 3.38 | >3.log₁₀ |
| | acid in 70% ethanol/water | | | |
| D | 4% tartaric acid in 70% | 2.56 | 3.0 | >3 log₁₀ |
| | ethanol/water | | | |

The clean fingerpads of the test subjects were treated with Samples A-D. Baseline skin pH readings were measured from the fingerpads prior to treatment with a composition. Skin pH measurements also were taken immediately after the composition dried on the fingerpads, and again after two hours.

All Samples A-D suppressed skin pH to below 4 for two hours. The combination of citric acid and malic acid (Sample C) maintained a lower pH at two hours than the same acids used singly (Samples A and B). The 4% tartaric acid composition (Sample D) showed the greatest suppression of skin pH.

Two hours after treatment of the fingerpads with the solutions, Rhinovirus 39 at a titer of 4×10⁴ pfu was applied to fingerpads. The virus was dried on the fingerpads for 10 minutes, then the fingerpads were rinsed with a viral recovery broth containing 75% EBSS and 25% FBS with 1× antibiotics. The sample was serially diluted in viral recovery broth and plated onto H1-HeLa cells. Titers were assayed as per the plaque assay. Complete inactivation of Rhinovirus 39 was achieved resulting in a greater than 3 log reduction.

The following examples illustrate that polymeric acids, and especially an acrylic acid homopolymer or copolymer, in the presence of alcohol impart antiviral efficacy. The polymeric acids have a low pH and good substantivity to skin, which effectively maintains a low skin pH over time, and helps provide a persistent antiviral efficacy. The polymeric acids also help provide an essentially continuous layer or film of an organic acid on treated surfaces, which in turn enhances the persistent antiviral activity of the composition.

A synergistic effect on the lowering of skin pH was demonstrated with using acrylic acid-based polymer in the presence of alcohol. However, an acrylic acid-based polymer in the absence of an alcohol did not maintain a reduced skin pH to the same degree over time. Importantly, skin pH reduction is less dependent on composition pH when a polymeric acid is used in conjunction with an alcohol. The synergy demonstrated between the polymeric acid and the alcohol was unexpected and is a novel way of providing the lowered skin pH that provides a desired persistent antiviral efficacy.

A synergistic effect on a rapid and persistent antiviral activity also is demonstrated when an acrylic acid-based polymer is used in conjunction with polycarboxylic acids. It has been found that utilizing a low amount of a polymeric acid (e.g., about 0.1% to about 2%, by weight) together with a polycarboxylic acid, like citric acid, malic acid, tartaric acid, and mixtures thereof, enhances the antiviral activities of the polycarboxylic acids. This synergistic effect allows a reduction in the polycarboxylic acid concentration in an antiviral composition, without a concomitant decrease in antiviral efficacy. This reduction in polycarboxylic acid concentration improves composition mildness by reducing the irritation potential of the composition. It is theorized, but not relied upon herein, that the polymeric acid assists in forming a residual barrier film or layer of organic acids on a treated surface, which enhance the persistent antiviral activity of the composition.

### EXAMPLE 7

A composition containing a polyacrylic acid (1% by wt), i.e., ULTREZ 20, available from Noveon Europe, was prepared in 70% aqueous ethanol and in water. Each composition (1.8 ml) was applied to the thumb, index, and middle fingers of a test subject. Skin pH readings were measured prior to treatment (baseline), immediately after the fingers were dry, and again after two hours. The average skin pH readings are summarized below.

| | Average skin pH | | | Viral |
|---|---|---|---|---|
| | Baseline | T = 0 | T = 2 hrs. | log 10 reduction |
| 70% ethanol | 5.65 | 5.3 | 5.2 | <0.2 |
| Polyacrylic acid (1%) | 5.63 | 4.4 | 4.5 | 1.8 |
| (70% aqueous ethanol) | | | | |
| Polyacrylic acid (1%) | 5.64 | 4.5 | 4.7 | 1.5 |
| (water) | | | | |

The polyacrylic acid suppressed skin pH to about 4.5 initially, and skin pH remains under 5 after two hours. The composition with ethanol suppressed skin pH slightly lower (4.4) than the composition free of ethanol (4.5). This result suggests a synergistic effect on lowering skin pH when a polyacrylic acid is applied with ethanol.

Two hours after treatment of the fingerpads with the above compositions, Rhinovirus 39 was applied to the fingerpads that had been treated at a titer of 9.8×10² pfu. The virus was dried on the fingerpads for 10 minutes, then the fingerpads were rinsed with viral recovery broth. The broth was serially diluted in viral recovery broth and plated onto H1-HeLa cells. Titers were assayed as per the plaque assay. Both compositions reduced the viral titer. However, the composition containing ethanol exhibited slightly greater efficacy against Rhinovirus by reducing the titer by 1.8 log versus 1.5 log for the composition without ethanol.

This data illustrates that polyacrylic acid suppresses skin pH resulting in antiviral efficacy. The data also illustrates that polyacrylic acid and ethanol act synergistically to lower skin pH, thus resulting in a greater efficacy against rhinovirus.

To demonstrate this efficacy, the following eight compositions were prepared, wherein solutions containing a polyacrylic acid (with and without ethanol) were buffered to a pH of about 4.5, 5.0, 5.5, or 6.0.

| Sam- | | Solution | Avg. Skin | Viral Log₁₀ |
|---|---|---|---|---|
| ple | Composition (by wt %) | pH | pH 2 hrs. | Reduction |
| A | 1% ULTREZ 20/70% ethanol | 4.54 | 4.52 | >2 log₁₀ |
| B | 1% ULTREZ 20/70% ethanol | 5.10 | 4.87 | >2 log₁₀ |
| C | 1% ULTREZ 20/70% ethanol | 5.54 | 4.41 | >2 log₁₀ |
| D | 1% ULTREZ 20/70% ethanol | 6.17 | 4.32 | >2 log₁₀ |
| E | 1% ULTREZ 20 | 4.57 | 4.93 | <1 log₁₀ |
| F | 1% ULTREZ 20 | 5.12 | 5.46 | <1 log₁₀ |
| G | 1% ULTREZ 20 | 5.55 | 5.33 | <1 log₁₀ |
| H | 1% ULTREZ 20 | 6.32 | 5.70 | <1 log₁₀ |

The effect of the eight compositions on both skin pH and viral efficacy was tested. Each composition (1.8 ml) was applied to the thumb, index, and middle fingers of a test subject. Skin pH readings were measured prior to treatment (baseline), immediately after the product had dried, and again after two hours.

The skin pH data indicated that a polyacrylic acid and ethanol function synergistically to suppress skin pH because each composition containing ethanol in combination with the polyacrylic acid suppressed skin pH to a lower value than compositions free of ethanol. Compositions containing ethanol and polyacrylic acid lowered skin pH to between pH 4 and 5 independent of the solution pH. In contrast, compositions free of ethanol suppress the skin pH only to between pH 5-6 and the final skin pH is similar to the solution pH.

To test the viral efficacy of the above compositions, Rhinovirus 39 at a titer of 1.7×10³ pfu was applied to the fingerpads after two hours. The virus dried for 10 minutes, eluted and diluted serially in viral recovery broth. Samples were plated on H1-HeLa cells, and virus titer was assayed as per the plaque assay method. The compositions containing ethanol in combination with polyacrylic acid had a greater than 2 log reduction in viral titers, whereas compositions free of ethanol exhibited a less than 1 log reduction in viral titers. Therefore, a synergism exists between polyacrylic acid and ethanol in reducing skin pH, which provides greater antiviral efficacy against rhinovirus. It is theorized, but not relied upon herein, that the ethanol helps provide a more continuous film or layer of the organic acid on the skin, for example, by reducing the surface tension of the composition for a more even and uniform application of the composition to a surface, and particularly skin.

### EXAMPLE 8

The following compositions were prepared to further illustrate the antiviral efficacy provided by a polyacrylic acid.

| | Composition | Solution | Avg. Skin | % Hands |
|---|---|---|---|---|
| Sample | (by wt %) Thickeners | pH | pH 2 hrs. | with Virus |
| A | 1% polyacrylic acid | 4.21 | 4.7 | 63% |
| B | 5.5% CRODAFOS | 5.41 | 5.0 | 100% |
| | Acid¹⁾ | | | |
| C | 1.25% NATROSOL 250 HHR | 6.32 | 5.3 | 100% |
| | CS²⁾ | | | |

¹⁾CRODAFOS CS20 Acid is Ceteth-20 & Cetaryl Alcohol & Dicetyl Phosphate; and
²⁾NATROSOL 250 HHR CS is hydroxyethylcellulose.

Samples A-C (1.8 ml) were applied to the thumb, index, and middle fingers of clean hands. Skin pH readings were taken prior to treatment (baseline), immediately after the fingers were dry, and again after two hours for Samples A and B and after four hours for Sample C. The averages of the skin pH values are provided in the above table.

Sample A containing polyacrylic acid lowered the skin pH to the greatest extent with a final skin pH after two hours of pH 4.7. Neither Sample B nor Sample C lowered the skin pH below pH 5.0. This data indicates that polyacrylic acid has an ability to suppress skin pH and maintain a low skin pH for a least two hours.

The viral efficacy of Samples A-C against Rhinovirus 39 was also tested. A viral load of about 10³ pfu was spread over the thumb, index, and middle fingers of each treated hand and allowed to dry for 10 minutes. The fingers then were rinsed with viral recovery broth and samples were serially diluted and plated on H1-HeLa cells. Viral titers were measured using the plaque assay. For both Samples B and C, 100% of the hands were positive for rhinovirus, which indicates little efficacy of these compositions against rhinovirus. In contrast, Sample A demonstrated a viral efficacy because only 63% of the hands were found positive for rhinovirus.

### EXAMPLE 9

Example 7 demonstrated that a synergism exists between polyacrylic acid and ethanol, which results in suppression of skin pH and antiviral efficacy. The following compositions were prepared to examine the effectiveness of polycarboxylic acid blends and a single polycarboxylic acid composition, each in combination with polyacrylic acid and ethanol, on antiviral efficacy. A preferred antiviral composition contains the least amount of organic acid required to demonstrate a persistent antiviral efficacy.

The compositions were applied to the fingerpads of clean hands. After the indicated times, about 10³ to 10⁴ pfu of Rhinovirus 39 was applied to the hands and allowed to dry for 10 minutes. The virus was recovered by rinsing the hands with viral recovery broth. The samples then were diluted serially in viral recovery broth and plated on H1-HeLa cells. Viral titers were determined by plaque assay. The percentage of hands that were positive for rhinovirus is summarized below.

| | | | % of Hands Positive |
|---|---|---|---|
| Composition (by wt %) | Time | | for Rhinovirus |
| 70% ethanol | 15 | min. | 100% |
| 1% citric acid/1% malic acid/10% | 1 | hr. | 100% |
| ethanol/water | | | |
| 1% polyacrylic acid/4% citric acid/ | 4 | hrs. | 91% |
| 70% ethanol/water | | | |
| 1% polyacrylic acid/1% citric acid/1% | 4 | hrs. | 0% |
| malic acid/70%ethanol/water | | | |

A composition containing 70% ethanol alone was not effective as an antiviral composition. Citric acid (1%) and malic acid (1%) lost effectiveness against rhinovirus after one hour because 100% of the hands were found to be positive for rhinovirus. In contrast, when a composition containing 1% citric and 1% malic acids are applied to the hands in combination with polyacrylic acid and 70% ethanol, no virus was detected on the hands after four hours. A single acid (4% citric acid) in combination with a polyacrylic acid and ethanol was less effective against rhinovirus because 91% of hands were found to be positive for rhinovirus after four hours.

This data demonstrates that using a polyacrylic acid and ethanol allows the use of a lower concentration of polycarboxylic acid to achieve a desired antiviral efficacy. This improvement is attributed, at least in part, to forming a residual film or layer of the organic acids on the skin.

### EXAMPLE 10

The use of a polyacrylic acid and ethanol in a composition suppresses skin pH to a value below the solution pH, as demonstrated in Example 7. To test whether antiviral compositions containing citric acid, malic acid, polyacrylic acid, and ethanol can be buffered to a higher solution pH and still provide a skin pH at or below pH 4 to obtain a persistent antiviral activity, the following compositions were prepared.

| | | | Skin | Skin | |
|---|---|---|---|---|---|
| | | Solution | pH | pH | Viral |
| Sample | Composition (by wt %) | pH | Initial | 4 hrs. | Reduction |
| A | 1% ULTREZ 20/2% citric | 3.2 | 2.9 | 3.7 | >3 log₁₀ |
| | acid/2% malic acid/70% | | | | |
| | ethanol | | | | |
| B | 1% ULTREZ 20/2% citric | 4.34 | 3.4 | 3.7 | >3 log₁₀ |
| | acid/2% malic acid/70% | | | | |
| | ethanol | | | | |
| C | 1% ULTREZ 20/2% citric | 4.65 | 3.6 | 3.8 | >3 log₁₀ |
| | acid/2% malic acid/70% | | | | |
| | ethanol | | | | |

The compositions (1.8 mL) were applied to the thumb, index, and middle fingers of clean hands. Skin pH readings were measured prior to treatment (baseline), immediately after the fingers were dry, and again after four hours. The average of the skin pH values are plotted above.

Initial skin pH of skin treated with Samples A-C were suppressed to between pH 2.9 and 3.6, wherein the lower the solution pH, the lower the initial skin pH. However, after four hours, the skin pH for all three compositions was about pH 3.7. Consistent with previous examples, solution pH did not predict subsequent skin pH.

The viral efficacy of Samples A-C against Rhinovirus 39 also was tested. A viral load of about 10³ pfu was spread over the thumb, index, and middle fingers of each treated hand and allowed to dry for 10 minutes. The fingers then were rinsed with viral recovery broth and samples were diluted serially and plated on H1-HeLa cells. Viral titers were measured using the plaque assay. No virus was recovered from any of the hands indicating that all three Samples A-C have antiviral efficacy.

This data demonstrates than when citric acid and malic acid are utilized in a composition in combination with a polyacrylic acid and ethanol, the pH of the solution can be buffered to a higher, e.g., milder and safer, pH for application to the skin, while still retaining an ability to suppress skin pH and exhibit antiviral activity. This result also is attributed, at least in part, to the residual layer or film of organic acid that remains on the skin after evaporation of volatile composition ingredients.

The following tests demonstrate that a composition of the present invention provides an essentially continuous barrier layer of organic acid on a treated surface. In particular, the following tests show that a present composition resists rinsing from a treated surface, e.g., at least 50% of the nonvolatile composition ingredients (including the organic acid) remains on a treated surface after three rinsings, as determined from NMR and IR spectra. In addition, an effective antiviral amount of the nonvolatile composition ingredients remains on a treated surface after 10 rinsings, also determined using NMR and IR spectra.

In the following tests, an aqueous composition containing, by weight, 2% malic acid, 2% citric acid, 1% polyacrylic acid, 62% ethanol, and 0.5% hydroxyethylcellulose as a gelling agent (Composition A) was compared to an aqueous composition, containing 2% malic acid, 2% citric acid, and 62% ethanol (Composition B). The compositions were applied to a glass surface to provide a film. From infrared (IR) and nuclear magnetic resonance (NMR) spectra of the film taken after each rinse, it was determined that Composition B was completely rinsed from the surface after one rinsing with water. Composition B therefore failed to exhibit water resistance and failed to provide a film or layer of nonvolatile composition ingredients on the surface.

In contrast, IR and NMR spectra showed that Composition A provided a rinse-resistant film or layer of composition ingredients on the treated surface. The amount of composition ingredients that remained on the treated surface was reduced over the first three rinsings, then resisted further removal from the treated surface in subsequent rinses. The IR and NMR spectra showed that detectable and effective amounts of the nonvolatile composition ingredients remained on the treated surface after 10 water rinses.

Another test was performed to measure the contact angle of water on a surface. “Contact angle” is a measure of the wetting ability of water on a surface. In this test, Compositions A and B were applied to a glass surface and allowed to dry. Contact angle then was measured for glass treated with Compositions A and B, both unrinsed and rinsed, using deionized water. The contact angle of bare, i.e., untreated, glass also was measured as a control. The following table summarizes the results of the contact angle test.

| | Composi- | Composition | Composition | Composition | |
|---|---|---|---|---|---|
| | tion A | A | B | B | Bare |
| | Unrinsed | Rinsed | Unrinsed | Rinsed | Glass |
| Avg | 45.96 | 72.66 | 6.69 | 41.51 | 38.47 |
| Reading | | | | | |
| (degrees) | | | | | |
| Change in | | 26.7 | | 34.8 | |
| degrees | | | | | |
| % Change | | 58.1 | | 520.2 | |

The contact angle data shows that Composition A modifies the glass surface and provides a persistent barrier film or layer on the glass surface. The data also shows that Composition B is rinsed from the surface because the contact angle after rinsing of Composition B is essentially the same as that of bare glass.

Another test was performed to demonstrate metal ion uptake by a residual film of Composition A. In this test, films of Composition A were formed on glass, dried at least 4 hours, then exposed to solutions having a 0.5 M concentration of metal ions. Samples then were analyzed by SEM scan. The data in the following table shows that a film resulting from Composition A effectively binds several types of metal ions. It is theorized, but not relied upon, that this is a surface phenomenon because no mechanism for transporting metal ions into the film is known.

Composition A Films on Glass (Metal-Soaked & Deionized Water Rinsed)
(unless otherwise specified)

| Soaking Solution | EDS atomic % | EDS wt % |
|---|---|---|
| 0.56 wt % CaCl₂ in formula | 0.63% Ca | 1.71% Ca |
| on 316 SS-No Rinse | | |
| 0.1 M Ca on 316 SS | 0.13% Ca | 0.21% Ca |
| 0.5 M Ca on 316 SS | 0.34% Ca | 0.54% Ca |
| 0.5 M Ca w/ more rinsing on | 0.07% Ca | 0.12% Ca |
| 316 SS | | |
| 0.5 M Cu on 316 SS | 0.65% Cu | 1.59% Cu |
| 0.5 M Fe on Al 6061 | 0.41% Fe | 1.14% Fe |
| 0.5 M Zn on Al 6061 | 0.24% Zn | 0.90% Zn |
| Metal Coupon anzlysis | | |
| DI water on 316 SS | 0% Ca, 0% Cu, | 0% Ca, 0% Cu, |
| Fe compensated for in above | 0% Zn | 0% Zn |
| datum | | |
| DI water on Al 6061 | 0.07% Ca, 0.08% | 0.18% Ca, 0.29% |
| | Fe, 0.03% Cu | Fe, 0.11% Cu |
| | [from Al] | [from Al] |

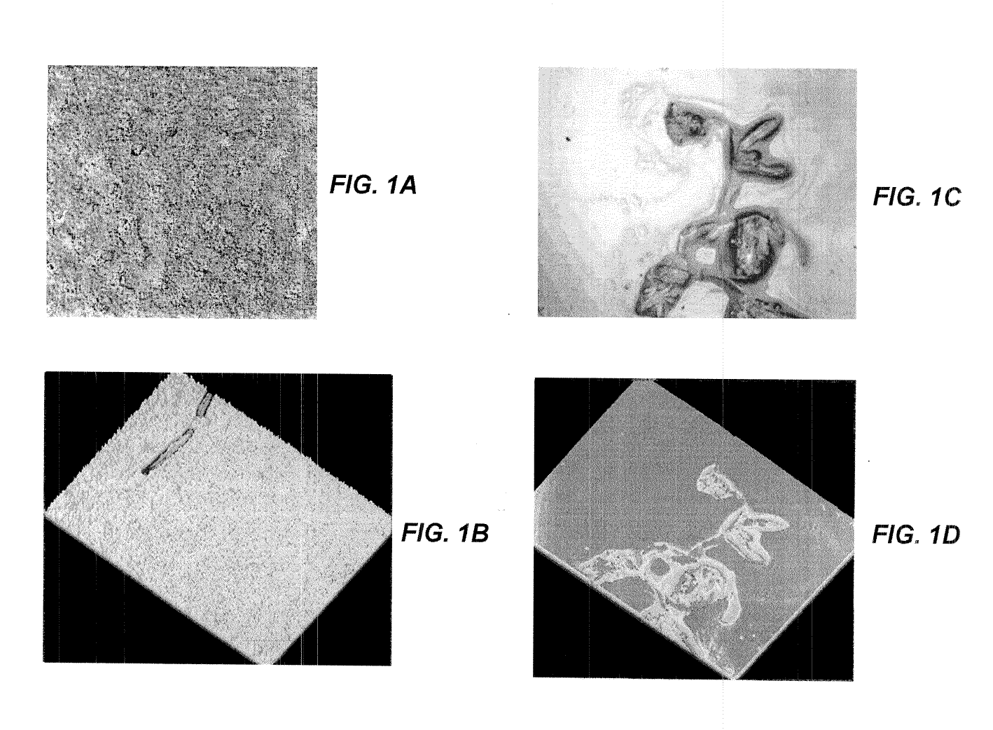

Reflectance micrographs showing the surface coverage of Compositions A and B also were taken (FIG. 1). The attached micrographs show that Composition A provides an essentially complete surface coverage, i.e., a more even coverage of Composition A on a treated surface, which provides an essentially continuous layer or film of nonvolatile composition ingredients on the surface. The attached micrographs are a digital conversion of reflectance values, which provide a direct correlation to surface coverage. The micrographs demonstrate that Composition A (FIGS. 1a) and 1b)) provides a film having improved adhesion, dispersion, and crystal formation compared to Composition B (FIGS. 1c) and 1d)).

### EXAMPLE 11

A time kill test was performed on additional bacteria to demonstrate the broad spectrum efficacy of a composition of the present invention. In this test, the following antimicrobial composition was tested.

| Ingredient | Weight Percent |
|---|---|
| Cetyl Alcohol | 1.00 |
| Glycerin | 1.00 |
| Isopropyl Palmitate | 1.00 |
| Dimethicone 100 CST | 1.02 |
| Ethanol SDA-40B | 3.09 |
| Natrosol 250 HHX | 0.26 |
| Deionized Water | 10.94 |
| Deionized Water | 17.65 |
| ULTREZ 10 Polymer | 1.01 |
| Ethanol SDA-40B | 58.82 |
| Citric Acid | 2.00 |
| Malic Acid | 2.00 |
| Sodium Hydroxide 50% | 0.22 |

The above-composition was tested for an ability to control the following microorganisms under the following conditions:

| | |
|---|---|
| Test Systems: | Staphylococcus aureus ATCC 6538 |
| | Escherichia coli ATCC 11229 |
| | Listeria monocytogenes ATCC 7644 |
| | Enterobacter cloacae ATCC 13047 |
| Test Temperature: | Ambient (20-25° C.) |
| Exposure Time: | 15 and 30 seconds |
| Neutralizer | 99 mL of D/E Broth |
| | A neutralizer screen performed as part of the testing |
| | verified that the neutralizer adequately neutralized |
| | the products and was not detrimental to the |
| | tested organisms. |
| Subculture Medium: | D/E Agar |
| Incubation: | 35 ± 2° C. for 48 ± 4 hours (for S. aureus, |
| | E. coli, L. monocytogenes) |
| | 30 ± 2° C. for 48 ± 4 hours (for E. cloacae) |
| | 26 ± 2° C. for 72 ± 4 hours (for C. albicans) |

The test data summarized are below:

### Inoculum Numbers (CFU/mL)

| Test System | A | B | Average |
|---|---|---|---|
| Staphylococcus aureus ATCC 6538 | 30 × 10⁶ | 29 × 10⁶ | 3.0 × 10⁷ |
| Escherichia coli ATCC 11229 | 18 × 10⁶ | 18 × 10⁶ | 1.8 × 10⁷ |
| Listeria monocytogenes ATCC 13047 | 26 × 10⁶ | 29 × 10⁶ | 2.8 × 10⁷ |
| Enterobacter cloacae ATCC 13047 | 31 × 10⁶ | 35 × 10⁶ | 3.3 × 10⁷ |

### Staphylococcus aureus ATCC 15442

| Exposure | | | | |
|---|---|---|---|---|
| Time | Survivors | Average | Log | Percent |
| (Seconds) | (CFU/mL) | Survivors (CFU/mL) | Reduction | Reduction |
| 15 | <100, <100 | <100 | >5.48 | >99.999 |
| 30 | <100, <100 | <100 | >5.48 | >99.999 |

### Escherichia coli ATCC 11229

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | 2 × 10², <100 | <1.5 × 10² | >5.08 | >99.999 |
| 30 | <100, <100 | <100 | >5.26 | >99.999 |

### Listeria monocytogenes ATCC 7644

| Exposure | | | | |
|---|---|---|---|---|
| Time | Survivors | Average Survivors | Log | Percent |
| (Seconds) | (CFU/mL) | (CFU/mL) | Reduction | Reduction |
| 15 | <100, 3 × 10² | <2.0 × 10² | >5.15 | >99.999 |
| 30 | <100, <100 | <100 | >5.45 | >99.999 |

### Enterobacter cloacae ATCC 13027

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, contamination | <100 | >5.52 | >99.999 |
| 30 | 5 × 10², 6 × 10² | 5.5 × 10² | 4.78 | >99.998 |

The data shows that a composition of present invention exhibits about a 4 to 5 log reduction at 15 and 30 seconds of exposure time against Staphylococcus aureus ATCC 6538, Escherichia coli ATCC 11229, Listeria monocytogenes ATCC 7644, and Enterobacter cloacae ATCC 13047.

### EXAMPLE 12

A composition of the invention containing an active antimicrobial agent, i.e., triclosan, is prepared by admixing the following ingredients at the indicated weight percentages until homogeneous.

| Ingredient | Weight Percent |
|---|---|
| Triclosan (TCS) | 0.15 |
| PPG-9 | 11.5 |
| Ethanol | 26 |
| Carbopol | 0.1 |
| Citric acid | 3 |
| Water | q.s. |

The pH of the composition is about 3.5. The composition has a percent saturation of TCS of 50%, and excellent antibacterial properties, exhibiting a greater than 3 log reduction in Gram positive and Gram negative bacteria in 30 seconds by the time kill test. The composition also eliminates norovirus from the skin.

### EXAMPLE 13

Another composition of the invention containing an active antimicrobial agent, i.e., salicylic acid, is prepared by admixing the following ingredients at the indicated weight percentages until homogeneous.

| Ingredient | Weight Percent |
|---|---|
| Triclosan (TCS) | 0.15 |
| PPG-9 | 11.5 |
| Ethanol | 26 |
| Carbopol | 0.1 |
| Salicylic acid | 1 |
| Water | q.s. |

The pH of the composition is about 3.5. The composition has a percent saturation of TCS of 50%, and an excellent antibacterial properties, exhibiting a greater than 3 log reduction in Gram positive and Gram negative bacteria in 30 seconds by the time kill test. The composition also eliminates human norovirus from the skin.

### EXAMPLE 14

This example was performed to determine the virucidal efficacy of a present composition against Feline Calicivirus, a surrogate for noroviruses known in the art. In this example, the following test method and method parameters were used.

Test Method:

ASTM E 1052-96: Standard Test Method for Efficacy of Antimicrobial Agents Against Viruses in Suspension

Method Parameters:

| | |
|---|---|
| Test System: | Feline Calicivirus, F-9 Strain, ATCC VR-782 |
| Organic Soil: | 5% Fetal Bovine Serum (FBS) |
| Exposure | Ambient (20-26° C.) |
| Temperature: | |
| Exposure Time: | 30 seconds |
| Neutralizer: | 100% Fetal Bovine Serum used to make |
| | 10⁻² dilution |
| Test Medium: | Minimum Essential Medium (MEM) supplemented |
| | with 5% heat inactivated FBS, 100 units/mL |
| | Penicillin, 10 μg/mL Gentamicin, 2.5 μg/mL |
| | Fungizone, 20 mM Hepes, and 2 mM Glutamine |
| Test Cell Cultures: | Crandell Reese Feline Kidney (CRFK) cells |
| Incubation: | 7 days at 35 ± 2° C., 5 ± 2% CO₂ |

The following four samples were tested for efficacy against Feline Calicivirus:

| Ingredient (wt. %) | A | B | C | D |
|---|---|---|---|---|
| Cetyl Alcohol | 1.00 | | | 1.00 |
| Glycerin | 1.00 | | | 1.00 |
| Isopropyl Palimitate | 1.00 | | | 1.00 |
| Dimethicone 100 CST | 1.02 | | | 1.02 |
| Ethanol SDA-40B | 3.09 | | | 3.09 |
| Natrosol 250 HHR | 0.26 | | | 0.26 |
| Deionized Water | 10.94 | 37.94 | 35.80 | 10.94 |
| Deionized Water | 17.65 | | | 17.65 |
| ULTREZ 10 Polymer | 1.01 | | | 1.01 |
| Ethanol SDA-40B | 58.82 | 59.83 | 60.00 | 58.82 |
| Citric Acid | 2.00 | 1.01 | 2.00 | 2.00 |
| Malic Acid | 2.00 | 1.00 | 2.00 | 2.00 |
| Sodium Hydroxide 50% | 0.22 | 0.23 | 0.20 | 0.22 |
| Total | 100.00 | 100.00 | 100.00 | 100.00 |
| pH | 3.51 | 3.54 | 3.50 | 3.51 |

General guidelines for assessing results based on EPA for virucides are (a) the product must demonstrate complete inactivation of the test virus at all dilutions; and (b) if cytotoxicity is present, at least a 3-log reduction in titer must be demonstrated beyond the cytotoxic level. For all Compositions A-D, the overall log reduction is reduced by the amount of cytotoxicity present.

In this test, Composition A displayed a complete inactivation of Feline Calicivirus after an exposure time of 30 seconds and is efficacious against this virus. The reduction in viral titer was ≧4.75 log₁₀.

Composition B displayed inactivation of Feline Calicivirus after an exposure time of 30 seconds and is efficacious against the virus. The reduction in viral titer was 3.0 log₁₀.

Composition C displayed a complete inactivation of Feline Calicivirus after an exposure time of 30 seconds and is efficacious against this virus. The reduction in viral titer was ≧5.75 log₁₀.

Composition D displayed a complete inactivation of Feline Calicivirus after an exposure time of 30 seconds and is efficacious against this virus. The reduction in viral titer was ≧4.75 log₁₀.

### EXAMPLE 15

A second test was performed to demonstrate the efficacy of a present antiviral composition against Feline Calicivirus. In this test, a composition of the present invention was compared to a present day commercial alcohol-based (60%) hand rub composition for antiviral activity.

In this test, Composition A of Example 14 was compared to a commercial alcohol hand rub composition, i.e., ENDURE 450, available from Ecolab, Inc., Eagan, Minn.

In this test, a suspension the virus was exposed to the use dilution of the test product. At each pre-determined exposure time, an aliquot was removed, neutralized by serial dilution, and assayed for the presence of virus. The positive virus controls, cytotoxicity controls, and neutralization controls were assayed in parallel. Antiviral properties of the product were evaluated and compared at the specified concentrations and time intervals.

Experimental Design:

| | |
|---|---|
| Dilution: | Ready to use (RTU) |
| Virus: | Feline Calicivirus, ATCC VR-782, Strain F-9 |
| Exposure Time: | 30 seconds, 1 minute, and 10 minutes |
| Exposure Temperature: | Room temperature (25.0° C.) |
| Test Medium: | Test medium used in this study was Minimum |
| | Essential Medium (MEM) supplemented with |
| | 5% heat inactivated FBS, 10 μg/mL |
| | Gentamicin, 100 units/mL penicillin, and |
| | 2.5 μg/mL amphotericin B. |
| Indicator Cell Cultures: | Feline Kidney (CRFK) |

In this test, the titer of the virus control was 7.25 log₁₀ following the 30 second, 1 minute, and 10 minute exposure times. Test substance cytotoxicity was observed at 2.5 log₁₀ for both Composition A and the Alcohol Hand Rub. The neutralization control demonstrated that Composition A was neutralized at ≦3.5 log₁₀.

Composition A demonstrated a ≧99.99% reduction in viral titer following a 30 second, 1 minute, and 10 minute exposure to Feline Calicivirus, compared to the virus control and taking the neutralization results into consideration. The log reduction in viral titer for all three exposure times was ≧4.0 log₁₀. The Alcohol Hand Rub demonstrated an 82.2% reduction in viral titer following a 30 second exposure to Feline Calicivirus compared to the virus control and taking the neutralization results into consideration. The log reduction in viral titer was 0.75 log₁₀. The Alcohol Hand Rub demonstrated a 99.7% reduction in viral titer following a 1 minute exposure to Feline Calicivirus compared to the virus control and taking the neutralization results into consideration. The log reduction in viral titer was 2.5 log₁₀. The Alcohol Hand Rub demonstrated a 99.97% reduction in viral titer following a 10 minute exposure to Feline Calicivirus compared to the virus control and taking the neutralization results into consideration. The log reduction in viral titer was 3.5 log₁₀.

Additional tests were performed to demonstrate the efficacy of a present composition against noroviruses. Example 16 demonstrate efficacy after repeated water rinses, and Example 17 illustrates the barrier properties provided by a composition of the present invention.

### EXAMPLE 16

This example shows the ability of a present composition, i.e., Composition A of Example 14, to retain efficacy over respected water rinsings. This example demonstrates that Composition A has an ability to form a film on a hard surface that retains antiviral properties after exposure to water rinsing. This experiment illustrates the ability of said film inactivate the nonenveloped virus, e.g., Feline Calicivirus, a recognized surrogate for norovirus by the United States Environmental Protection Agency, after the applied product has been rinsed with water several times.

In this example, one-square inch of Composition A was coated onto a glass microscope slide (1″×3″) and allowed to dry at room temperature, i.e., about 25° C. Each coupon was rinsed with 5 ml of purified water and allowed to dry. This procedure was repeated for a standard number of rinses. Ten microliters of Feline Calicivirus was inoculated onto product film and spread to cover entire surface. Initial inoculum levels were 10^{6.25}. Total exposure time was ten minutes, then the product was neutralized, diluted, and incubated for 7 days at 35° C.

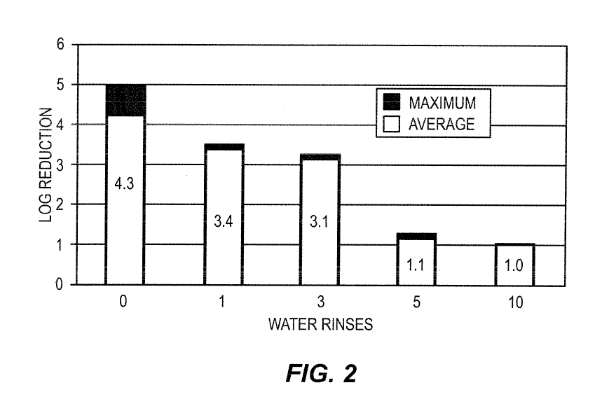

The results are summarized below, and in FIG. 2, showing that antiviral activity is maintained over 10 water rinses.

### Log Reduction Feline Calicivirus

| Number of Rinses | Sample 1 | Sample 2 | Average | fmax − Average |
|---|---|---|---|---|
| 0 | 3.5 | 5 | 4.3 | 0.8 |
| 1 | 3.5 | 3.3 | 3.4 | 0.1 |
| 3 | 3 | 3.3 | 3.1 | 0.1 |
| 5 | 1.3 | 1 | 1.1 | 0.1 |
| 10 | 1 | 1 | 1 | 0 |

### EXAMPLE 17

This example demonstrates that Composition A of Example 14 has an ability to form a film on a hard surface, which exhibits persistent antiviral properties to inactivate the nonenveloped virus, Feline Calicivirus, a recognized surrogate for norovirus by the United States Environmental Protection Agency.

In this example, one-square inch of Composition A was coated onto a glass microscope slide (1″×3″) and allowed to dry at room temperature for a given time. Ten microliters of Feline Calicivirus were inoculated onto product film and spread to cover entire surface. Initial inoculum levels were 10^{6.5} (Test One) and 10^{5.75} (Test Two). Total exposure time was ten minutes (Test One) or fifteen minutes (Test Two), then this product was neutralized, diluted, and incubated for 7 days at 35° C.

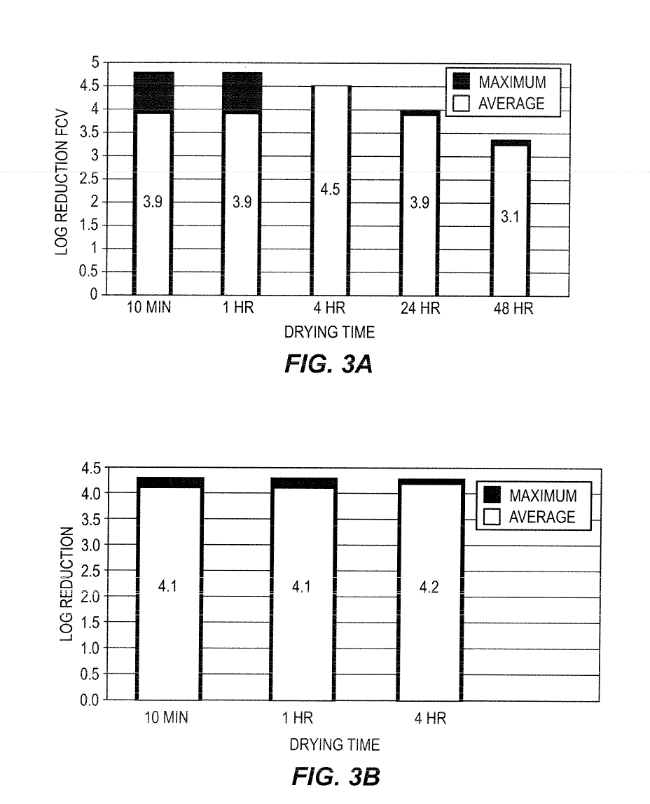

The results are summarized below and in FIG. 3a, show the ability of a barrier layer to control viruses.

### Log Reduction Feline Calicivirus 10 Minutes

| Dry Time | Sample 1 | Sample 2 | Average | fmax − Average |
|---|---|---|---|---|
| 10 min | 4.8 | 3.0 | 3.9 | 0.9 |
| 1 hr | 3.0 | 4.8 | 3.9 | 0.9 |
| 4 hr | 4.5 | | 4.5 | 0 |
| 24 hr | 3.8 | 4.0 | 3.9 | 0.1 |
| 48 hr | 3.0 | 3.3 | 3.1 | 0.1 |

Select data points were repeated in triplicate and the exposure time was adjusted out 15 minutes. The results are set forth below in FIG. 3b.

### Log Reduction Feline Calicivirus 15 Minutes

| Dry | | | | | |
|---|---|---|---|---|---|
| Time | Sample 1 | Sample 2 | Sample 3 | Average | fmax − Average |
| 10 min | 3.75 | 4.25 | 4.25 | 4.1 | 0.2 |
| 1 hr | 4 | 4.25 | 4 | 4.1 | 0.2 |
| 4 hr | 4.25 | 4.25 | 4 | 4.2 | 0.1 |

### EXAMPLE 18

This example demonstrates the residual virucidal efficacy of Composition A of Example 14 against Feline Calicivirus.

In this example, Composition A films were prepared by sectioning a one inch by one inch area on glass microscope slides with two thicknesses of cellophane tape. The slides were lined up, Composition A was added, and the product was run across the slides with a stainless steel spatula to create even product films. The tape then was removed and the films were allowed to dry for a specified amount of time.

After the dry time, ten microliters of Feline Calicivirus were inoculated onto the product film and spread across the film with a cell scraper. The films were incubated at room temperature in a biosafety cabinet for the specified exposure period. A cytotoxicity control was included by inoculating the product film with cell culture media instead of virus.

Thirty seconds before the end of the exposure period, 100 microliters of cell culture media were added to the film and a cell scraper was used to remove the product film from the slide. Two milliliters of fetal bovine serum were used to rinse the slide and neutralize the product. Each sample (considered the 10⁻² dilution) was vortexed with sterile glass beads and ten-fold serial dilutions were made in cell culture media.

Four wells of a cell seeded 24-well cell culture plate (each well already containing one milliliter of cell culture medium) were inoculated with 100 microliters of each dilution. The cell culture plates were incubated for seven days at 35±2° C., 5±2% CO₂.

A dried virus control was included by spreading ten microliters of Feline Calicivirus onto a glass slide and tested using the procedure listed above.

Product neutralization was confirmed by inoculating a low titer of Feline Calicivirus into two of the four wells for each dilution on the cytotoxicity plate.

| | |
|---|---|
| Test System: | Feline Calicivirus, F-9 Strain, ATCC VR-782 |
| Organic Soil: | 5% Fetal Bovine Serum |
| Exposure | Ambient (20-26° C.) |
| Temperature: | |
| Exposure Time: | 10 minutes (all dry times), 5 minutes (48 hours |
| | only) |
| Neutralizer: | 100% Fetal Bovine Serum |
| Test Medium: | Minimum Essential Medium (MEM) supplemented |
| | with 5% heat inactivated FBS, 100 units/mL |
| | Penicillin, 10 μg/mL Gentamicin, 2.5 μg/mL |
| | Fungizone, 20 mM Hepes, and 2 mM Glutamine |
| Test Cell Cultures: | Crandell Reese Feline Kidney (CRFK) cells |
| Incubation: | 7 days at 35 ± 2° C., 5 ± 2% CO₂ |

The residual activity of Composition A was demonstrated by the following results.

After 10 minutes: The reductions in the Feline Calicivirus viral titer were ≧4.75 and 3.0 log₁₀ after a 10 minute exposure time.

After 1 hour: The reductions in the Feline Calicivirus viral titer were 3.25 and ≧4.75 log₁₀ after a 10 minute exposure time.

After 4 hours: The reduction in the Feline Calicivirus viral titer was 4.5 log₁₀ after a 10 minute exposure time.

After 24 hours: The reductions in the Feline Calicivirus viral titer were 3.75 and ≧4.0 log₁₀ after a 10 minute exposure time.

After 48 hours: The reductions in the Feline Calicivirus viral titer for the 5 minute exposure time were 3.0 and 3.25 log₁₀. The reductions in the viral titer for the 10 minute exposure time were 4.25 and ≧3.25 log₁₀.

### EXAMPLE 19

The tests of Example 18 were repeated, but using an exposure time of 15 minutes. The residual activity of Composition A was demonstrated by the following results.

Composition A after 10 minutes: The reductions in the Feline Calicivirus viral titer were 3.75, ≧4.25, and ≧4.25 log₁₀ after a 15 minute exposure time.

Composition A after 1 hour: The reductions in the Feline Calicivirus viral titer were 4.0, ≧4.25, and 4.0 log₁₀ after a 15 minute exposure time.

Composition A after 4 hours: The reductions in the Feline Calicivirus viral titer were ≧4.25, ≧4.25, and 4.0 log₁₀ after a 15 minute exposure time.

### EXAMPLE 20

This example further demonstrates the residual efficacy of Composition A of Example 14 against Feline Calicivirus. In this example, films of Composition A were prepared by sectioning a one inch by one inch area on glass microscope slides with two thicknesses of cellophane tape. The slides were lined up, Composition A was added, and the product was run across the slides with a stainless steel spatula to create even product films. The tape was removed and the films were allowed to dry for 20 minutes. Using five milliliters of MilliQ water, the films were rinsed a specified number of times and allowed to dry for 20-30 minutes between rinses.

After the final dry time, ten microliters of Feline Calicivirus was inoculated onto the product film and spread across the film with a cell scraper. The films were incubated at room temperature in the biosafety cabinet for the specified exposure period. A cytotoxicity control was included by inoculating the product film with cell culture media instead of virus.

Thirty seconds before the end of the exposure period, 100 microliters of cell culture media was added to the film and a cell scraper was used to remove the product film from the slide. Two milliliters of fetal bovine serum were used to rinse the slide and neutralize the product. Each sample (considered the 10⁻² dilution) was vortexed with sterile glass beads and ten-fold serial dilutions were made in cell culture media.

Four wells of a cell seeded 24-well cell culture plate (each well already containing one milliliter of cell culture medium) were inoculated with 100 microliters of each dilution. The cell culture plates were incubated for seven days at 35±2° C., 5±2% CO₂.

A dried virus control was included by spreading ten microliters of Feline Calicivirus onto a glass slide and tested using the procedure listed above.

Product neutralization was confirmed by inoculating a low titer of Feline Calicivirus into two of the four wells for each dilution on the cytotoxicity plate.

| | |
|---|---|
| Test System: | Feline Calicivirus, F-9 Strain, ATCC VR-782 |
| Organic Soil: | 5% Fetal Bovine Serum |
| Exposure | Ambient (20-26° C.) |
| Temperature: | |
| Exposure Time: | 10 minutes |
| Neutralizer: | 100% Fetal Bovine Serum |
| Test Medium: | Minimum Essential Medium (MEM) supplemented |
| | with 5% heat inactivated FBS, 100 units/mL |
| | Penicillin, 10 μg/mL Gentamicin, 2.5 μg/mL |
| | Fungizone, 20 mM Hepes and 2 mM Glutamine |
| Test Cell Cultures: | Crandell Reese Feline Kidney (CRFK) cells |
| Incubation: | 7 days at 35 ± 2° C., 5 ± 2% CO₂ |

The residual activity of Composition A was demonstrated by the following results.

Composition A after 0 Rinses: The reductions in the Feline Calicivirus viral titer were 3.5 and ≧5.0 log₁₀ after a 10 minute exposure time.

Composition A after 1 Rinse: The reductions in the Feline Calicivirus viral titer were 3.5 and 3.25 log₁₀ after a 10 minute exposure time.

Composition A after 3 Rinses: The reductions in the Feline Calicivirus viral titer were 3.0 and 3.25 log₁₀ after a 10 minute exposure time.

Composition A after 5 Rinses: The reductions in the Feline Calicivirus viral titer were 1.25 and 1.0 log₁₀ after a 10 minute exposure time.

Composition A after 10 Rinses: The reduction in the Feline Calicivirus viral titer was 1.0 log₁₀ after a 10 minute exposure time.

### EXAMPLE 21

This example demonstrates the efficacy of a present antimicrobial composition in controlling norovirus using the fingerpads of adult volunteers. In particular, this example shows the ability of Composition A of Example 14 to reduce the level of Feline Calicivirus, ATCC VR-782 on human fingerpads 30 and 60 seconds, and 2 and 4 hours, after treatment.

In this test, twelve subjects completed the study. Eight subjects completed the study using Composition A of Example 14, and four subjects completed the study using ENDURE 450, a commercial alcohol-based sanitizing composition available from Ecolab, Inc., Eagan, Md. Three fingerpads per hand were used for treatment (test article) and one fingerpad per hand of each subject was used as the untreated control. Treated fingerpads and untreated control fingerpads were randomized among the eight digits. One thumb per subject was used as the “input” control (virus counts before drying). Two subjects completed the neutralizer effectiveness phase of the study.

For Composition A, one male and seven female subjects completed the study. For ENDURE 450, two male and two female subjects completed the study. Nine subjects were excluded or withdrew from the study. Four subjects were enrolled in the conditioning phase of the neutralization study. Two subjects, one male and one female, who met the study criteria were enrolled and completed the neutralization study. Two subjects withdrew from the neutralization phase of the study.

The data was evaluated using parametric statistical analysis as follows. The log₁₀ count of twelve viral recoveries for Composition A treatment was averaged. The changes from the average of the untreated counts (fifteen viral recoveries) were obtained for the test article at each time period (30 seconds, 60 seconds, 2 hours and 4 hours). Both log₁₀ and percent reductions were calculated.

Similarly, the log₁₀ count of six viral recovered for ENDURE 450 was averaged. The changes from the average of the untreated counts (eight viral recoveries) were obtained for the test article at each time period (30 seconds, 60 seconds, 2 hours and 4 hours). Both log₁₀ and percent reductions were calculated.

Composition A showed more than a 1.5 log reduction in the titer of Feline Calicivirus after 30 seconds of exposure to the composition and more than a 1.9 log reduction in viral titer after 60 seconds of exposure to the composition. After 2 and 4 hours from the initial treatment with Composition A, a more than 1.7 log reduction in viral titer could be shown when the virus was applied to the fingerpad. These results demonstrate a persistent antiviral effect of Composition A on the fingerpad for up to 4 hours after treatment.

The comparative ENDURE 450 composition showed a more than 1.7 log reduction in the titer of Feline Calcivirus after 30 seconds of exposure to the product, and a more than 2 log reduction in viral titer after 60 seconds of exposure to the product. After 2 and 4 hours from the initial treatment with ENDURE 450, a much smaller reduction in viral titer (0.17 at 2 hours and 0.4 at 4 hours) was shown when the virus was applied to the fingerpad. Overall, this data shows a greater effect with respect to antiviral persistence using Composition A on fingerpads inoculated with Feline Calicivirus than ENDURE 450.

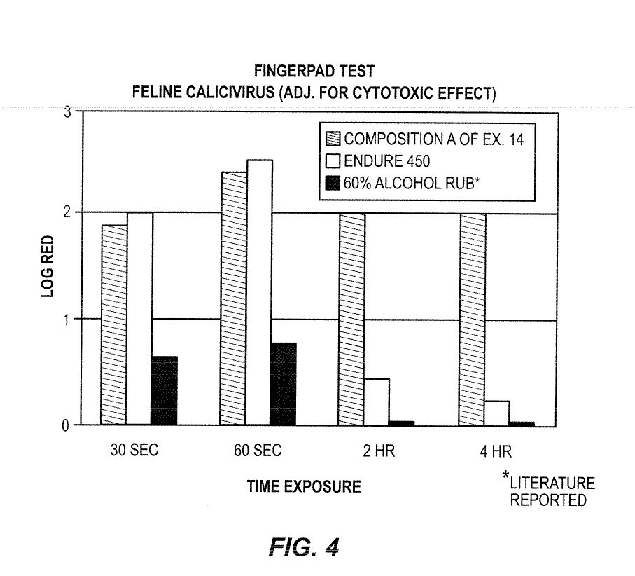

The test results are summarized in FIG. 4 showing a substantial residual activity for Composition A of 99% reduction over 4 hours. FIG. 4 also contains literature data for a straight 60% alcohol rub showing essentially no residual activity.

### EXAMPLES 22-25

| | Example | Example | Example | Example |
|---|---|---|---|---|
| | 22 | 23 | 24 | 25 |
| Ethanol SDA 40B 190 | 75 | 85 | 95 | 25 |
| Proof | | | | |
| Octanoic Acid | | 0.05 | | 0.05 |
| Citric Acid | 0.5 | 0.5 | 1.5 | 0.5 |
| Malic Acid | 0.5 | 0.5 | 1.5 | 0.5 |
| Pluronic F108 | | 0.2 | 0.2 | 0.2 |
| Sodium Hydroxide or | qs | qs | qs | qs |
| buffer | | | | |
| Deionized Water | 24 | 13.75 | 1.8 | 73.75 |
| Total | 100 | 100 | 100 | 100 |

All compositions of Examples 22-25 are clear and colorless, and leave a slight residue when sprayed onto a countertop and allowed to dry.

The antimicrobial compositions of the present invention have several practical end uses, including hand cleansers, surgical scrubs, body splashes, antiseptics, disinfectants, hand sanitizer gels, deodorants, dental care additives, and similar personal care products. Additional types of compositions include foamed compositions, such as mousses and the like, and compositions containing organic and inorganic filler materials, such as emulsions, lotions, ointments, creams, pastes, and the like. The compositions further can be used as an antimicrobial for inanimate surfaces, for example, sinks and countertops in hospitals, cruise ships, nursing homes, food service areas, and meat processing plants. The present antimicrobial compositions can be manufactured as dilute ready-to-use compositions, or as concentrates that are diluted prior to use.

As discussed above, both animate and inanimate surfaces can be treated in accordance with the method of the present invention. A particularly important surface is mammalian skin, and particularly human skin, to inactivate and interrupt the transmission of bacteria and noroviruses. However, the present method also is useful in treating inanimate surfaces of all types.

The present method is useful to treat hard surfaces. As used herein with respect to the surfaces treated by the present compositions, the term “hard” refers to surfaces comprising refractory materials, such as glazed and unglazed tile, brick, porcelain, ceramics, metals, glass, and the like, and also includes wood and hard plastics such as formica, polystyrenes, vinyls, acrylics, polyesters, and the like. Such surfaces are found, for example, in kitchens and bathrooms. A hard surface can be porous or nonporous.

The present method also can be used to treat hard surfaces in processing facilities (such as dairy, brewing, and food processing facilities), healthcare facilities (such as hospitals, clinics, surgical centers, dental offices, and laboratories), long-term healthcare facilities (such as nursing homes), farms, cruise ships, schools, and private homes.

The present method can be used to treat environmental surfaces such as floors, walls, ceilings, and drains. The method can be used to treat equipment such as food processing equipment, dairy processing equipment, brewery equipment, and the like. The compositions can be used to treat a variety of surfaces including food contact surfaces in food, dairy, and brewing facilities, countertops, furniture, sinks, and the like. The method further can be used to treat tools and instruments, such as medical tools and instruments, dental tools and instruments, as well as equipment used in the healthcare industries and institutional kitchens, including knives, wares (such as pots, pans, and dishes, cutting equipment, and the like. Methods of treating hard surfaces are described in U.S. Pat. Nos. 5,200,189; 5,314,687; and 5,718,910, the disclosures of which are incorporated herein by reference in their entirety.

In addition to the hard surfaces, the method can be used to treat textiles, such as clothing, protective clothing, laboratory clothing, surgical clothing, patient clothing, carpets, bedding, towels, linens, and the like.

In use, the compositions are applied to contact a target surface. The surface can be animate or inanimate. The compositions can be applied by dipping a surface into the composition, soaking a surface in the composition, or spraying, wiping, brushing, foaming, misting, rolling, pad coating, mopping, sponging, or fogging the composition onto a surface. The compositions can be applied manually, using equipment, such as a spray bottle, or by machine, such as a spray machine, foam machine, and the like. The compositions also can be used inside a machine, such as a warewashing machine or laundry machine.

Treatable inanimate surfaces include, but are not limited to, exposed environmental surfaces, such as tables, floors, walls; kitchenwares, including pots, pans, knives, forks, spoons, and plates; food cooking and preparation surfaces, including dishes; food preparation equipment; and tanks, vats, lines, pumps, hoses, and other process equipment. One useful application of the composition is to dairy processing equipment, which is commonly made from glass or stainless steel. Such equipment can be found both in dairy farm installations and in dairy plant installations for the processing of milk, cheese, ice cream, and other dairy products.

The method of the present invention also can be used in the manufacture of beverages, including fruit juice, dairy products, malt beverages, bottled water products, teas, and soft drinks. The method can be used to treat pumps, lines, tanks, and mixing equipment used in the manufacture of such beverages. The method of the present invention also can be used to treat air filters.

The method of the present invention also can be used to treat medical carts, medical cages, and other medical instruments, devices, and equipment. Examples of medical apparatus treatable by the present method are disclosed in U.S. Pat. No. 6,632,291, incorporated herein by reference.

For household applications, hand-operated pump-type or pressurized aerosol sprayers can be used. The compositions also can be employed to coat or otherwise treat materials such as sponges, fibrous or nonfibrous web materials, swabs, flexible plastics, textiles, wood, and the like. Generally, the coating process is used to impart prolonged antiviral properties to a hard porous or nonporous surface by coating said surface with the composition. The compositions also can be incorporated into a web material to provide an antimicrobial wiping article. The wiping article can be used to sanitize animate or inanimate surfaces.

An antimicrobial composition of the present invention can be formulated into a variety of product forms, including liquids, gels, and semisolids. The liquid product form can be a solution, dispersion, emulsion, or a similar product form. Gel and semisolid product forms can be transparent or opaque, designed for application by stick dispenser or by the fingers, for example. The present antimicrobial compositions can be manufactured as dilute ready-to-use compositions, or as concentrates that are diluted prior to use.

One particular product form is a liquid or solid composition disposed within a water-soluble packet. The packet is added to a proper amount of water, and the composition is released when the packet dissolves. The water-soluble packet typically comprises a polyvinylalcohol. One form of water-soluble packet is disclosed in U.S. Pat. No. 5,316,688, incorporated herein by reference. Numerous other water-soluble packets are known to person skilled in the art, for example, in U.S. Pat. Nos. 5,070,126; 6,608,121; and 6,787,512; U.S. Patent Publication No. 2002/0182348; WO 01/79417; and European Patent Nos. 0 444 230, 1 158 016, 1 180 536, and 1 251 147, each incorporated herein by reference. Capsules are another related and useful product form.

Yet another product form is incorporation of the composition into an absorbent or adsorbent carrier, such as polymeric microparticles or inorganic particles. The loaded carrier can be used as is, or incorporated into other product forms, either liquid, gel, semisolid, or solid.

Still another product form is a web material or swab containing an antimicrobial composition. The composition then can be applied to the skin by wiping the surface with the web material containing the composition.

Another product form is an article, such as latex gloves, having the composition applied to, or imbedded into, the article. During use, the composition imparts antiviral activity to the article itself and/or to a surface contacted by the article. Additional articles that can have an active composition imbedded therein are plastic cups, food wraps, and plastic containers.

The present invention, therefore, encompasses applying an effective amount of an antimicrobial composition of the present invention onto inanimate surfaces, such as household surfaces, e.g., countertops, kitchen surfaces, food preparing surfaces (cutting boards, dishes, pots and pans, and the like); major household appliances, e.g., refrigerators, freezers, washing machines, automatic dryers, ovens, microwave ovens, and dishwashers; cabinets; walls; floors; bathroom surfaces, shower curtains, garbage cans, and/or recycling bins, and the like.

In one embodiment of the present invention, a person suffering from a norovirus infection, or who is likely to be exposed to other individuals suffering from a norovirus infection, can apply a present antimicrobial composition to his or her hands. This application kills bacteria and inactivates norovirus particles present on the hands. The applied composition, either rinsed off or allowed to remain on the hands, preferably provides a persistent antiviral activity. Norovirus particles therefore are not transmitted to noninfected individuals via hand-to-hand transmission. The amount of the composition applied, the frequency of application, and the period of use will vary depending upon the level of disinfection desired, e.g., the degree of microbial contamination and/or skin soiling.

The present antimicrobial compositions provide the advantages of a broad spectrum kill of Gram positive and Gram negative bacteria, and a norovirus control, in short contact times. The short contact time for a substantial log reduction of bacteria is important in view of the typical 15 to 60 second time frame used to sanitize the skin and inanimate surfaces. The composition preferably imparts a persistent antiviral activity to the contacted surface.

The present compositions are effective in a short contact time because of the reduced pH of the composition, and the synergistic effect provided by the combination of a disinfecting alcohol and an organic acid, and a persistent activity is enhanced because of a residual barrier layer or film of composition ingredients that can remain on the skin after evaporation of the volatile components of the composition.

The present compositions further are effective in a short contact time in embodiments containing an optional active antimicrobial agent because the antimicrobial agent is present in the aqueous continuous phase of the present compositions, as opposed to surfactant micelles, and because of the reduced pH of the composition. The antimicrobial agent, therefore, is available to immediately begin reducing bacterial populations, and further is available to deposit on the skin to provide persistent antimicrobial efficacy. In addition, because the antimicrobial agent is in solution as opposed to surfactant micelles, the absolute amount of antimicrobial agent in the composition can be reduced without adversely affecting efficacy, and the antimicrobial agent is not rinsed from the skin with the surfactant prior to performing its antimicrobial function.

Obviously, many modifications and variations of the invention as hereinbefore set forth can be made without departing from the spirit and scope thereof, and, therefore, only such limitations should be imposed as are indicated by the appended claims.

What is claimed is:

1. A method of reducing a norovirus population on a surface comprising contacting the surface with a composition for 30 seconds to achieve a log reduction of at least 3 against the norovirus,
said composition comprising
(a) about 25% to about 95%, by weight, of a disinfecting alcohol;
(b) a virucidally effective amount of an organic acid;
(c) about 0% to about 5%, by weight, of an active antimicrobial agent;
(d) 0% to about 5%, by weight, of a gelling agent; and
(e) water,
wherein the composition has a pH of about 5 or less at 25° C.

2. The method of claim 1 wherein the composition forms a barrier layer comprising the organic acid on the surface.

3. The method of claim 1 wherein an essentially continuous layer comprising the organic acid is formed on the surface.

4. The method of claim 1 wherein the norovirus comprises a GI, GII, or GIV genogroup.

5. The method of claim 1 further comprising rinsing the composition from the surface.

6. The method of claim 1 wherein the composition is allowed to remain on the surface and dry.

7. The method of claim 1 wherein the surface is a skin of a mammal.

8. The method of claim 7 wherein the composition lowers a pH of skin to less than 4 after drying on the skin.

9. The method of claim 1 wherein the surface is an inanimate surface.

10. The method of claim 9 wherein the inanimate surface is a food contact surface.

11. The method of claim 1 wherein the composition imparts a persistent activity against norovirus to the surface.

12. The method of claim 1 wherein the disinfecting alcohol is present in the composition in an amount of about 30% to about 75%, by weight.

13. The method of claim 1 wherein the disinfecting alcohol is a C₁₋₆ alcohol or mixtures thereof.

14. The method of claim 1 wherein the disinfecting alcohol is selected from the group consisting of methanol, ethanol, isopropyl alcohol, n-butanol, n-propyl alcohol, and mixtures thereof.

15. The method of claim 1 wherein the composition comprises about 0.05% to about 15%, by weight, of the organic acid.

16. The method of claim 1 wherein the organic acid in the composition has a log P of less than one.

17. The method of claim 1 wherein the organic acid in the composition has a log P of one or greater.

18. The method of claim 1 wherein the organic acid comprises a first organic acid having a log P of less than one and a second organic acid having a log P of one or greater.

19. The method of claim 1 wherein the organic acid has a water solubility of at least about 0.05% by weight, at 25° C.

20. The method of claim 1 wherein the organic acid comprises a monocarboxylic acid, a polycarboxylic acid, a polymeric acid having a plurality of carboxylic, phosphate, sulfonate, and/or sulfate moieties, anhydrides thereof, or mixtures thereof.

21. The method of claim 1 wherein the organic acid comprises a monocarboxylic acid having a structure RCO₂H, wherein R is C₁₋₁₀alkyl, hydroxyC₁₋₃alkyl, haloC₁₋₃alkyl, phenyl, or substituted phenyl.

22. The method of claim 20 wherein the monocarboxylic acid is selected from the group consisting of acetic acid, propionic acid, octanoic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof.

23. The method of claim 1 wherein the organic acid comprises a polycarboxylic acid containing two to four carboxylic acid groups, and optionally contains one or more hydroxyl group, amino group, or both.

24. The method of claim 23 wherein the polycarboxylic acid is selected from the group consisting of malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

25. The method of claim 1 wherein the organic acid comprises a polymeric acid having a molecular weight of about 500 to about 10,000,000 g/mol.

26. The method of claim 20 wherein the polymeric acid is water soluble or water dispersible.

27. The method of claim 25 wherein the polymeric acid is selected from the group consisting of a polymeric carboxylic acid, a polymeric sulfonic acid, a sulfated polymer, a polymeric phosphoric acid, and mixtures thereof.

28. The method of claim 20 wherein the polymeric acid comprises a homopolymer or a copolymer of acrylic acid.

29. The method of claim 1 wherein the organic acid comprises an anhydride of a polycarboxylic acid.

30. The method of claim 20 wherein the organic acid comprises a polycarboxylic acid and a polymeric carboxylic acid.

31. The method of claim 30 wherein the polycarboxylic acid comprises citric acid, malic acid, tartaric acid, or mixtures thereof, and the polymeric carboxylic acid comprises a homopolymer or a copolymer of acrylic acid, or methacrylic acid.

32. The method of claim 31 wherein the polymeric acid comprises a homopolymer or a copolymer of acrylic acid.

33. The method of claim 1 wherein the composition comprises about 0.01% to about 2%, by weight, of the active antimicrobial agent.

34. The method of claim 33 wherein the active antimicrobial agent comprises a phenolic antimicrobial agent selected from the group consisting of:
(a) a 2-hydroxydiphenyl compound having the structure 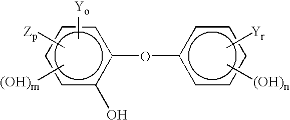
wherein Y is chlorine or bromine, Z is SO₃H, NO₂, or C₁-C₄ alkyl, r is 0 to 3, o is 0 to 3, p is 0 or 1, m is 0 or 1, and n is 0 or 1;
(b) a phenol derivative having the structure 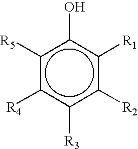
wherein R₁ is hydro, hydroxy, C₁-C₄ alkyl, chloro, nitro, phenyl, or benzoyl, R₂ is hydro, hydroxy, C₁-C₆ alkyl, or halo, R₃ is hydro, C₁-C₆ alkyl, hydroxy, chloro, nitro, or a sulfur in the form of an alkali metal salt or ammonium salt, R₄ is hydro or methyl, and R₅ is hydro or nitro;
(c) a diphenyl compound having the structure 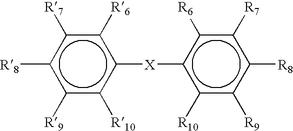
wherein X is sulfur or a methylene group, R₆ and R′₆ are hydroxy, and R₇, R′₇, R₈, R′₈, R₉, R′₉, R₁₀, and R′₁₀, independent of one another, are hydro or halo; and
(d) mixtures thereof.

35. The method of claim 33 wherein the antimicrobial agent comprises triclosan, p-chloro-m-xylenol, hydrogen peroxide, benzyl peroxide, benzyl alcohol, a quaternary ammonium compound, or a mixture thereof.

36. The method of claim 33 wherein the antimicrobial agent is present in the composition in an amount of at least 50% of saturation concentration, when measured at 25° C.

37. The method of claim 36 wherein the antimicrobial agent is present in an amount of at least 75% of saturation concentration.

38. The method of claim 37 wherein the antimicrobial agent is present in an amount of at least 95% of saturation concentration.

39. The method of claim 1 wherein the gelling agent is present in the composition in an amount of about 0.1% to about 3%, by weight.

40. The method of claim 39 wherein the gelling agent comprises a natural gum, a synthetic polymer, a clay, an oil, a wax, and mixtures thereof.

41. The method of claim 39 wherein the gelling agent is selected from the group consisting of cellulose, a cellulose derivative, guar, a guar derivative, algin, an algin derivative, a water-insoluble C₈-C₂₀ alcohol, carrageenan, a smectite clay, a polyquatemium compound, and mixtures thereof.

42. The method of claim 1 wherein the composition is free of a surfactant.

43. The method of claim 1 wherein the composition further comprises about 0.1% to about 15%, by weight, of a surfactant.

44. The method of claim 44 wherein the surfactant comprises an anionic, cationic, nonionic, or ampholytic surfactant, or mixtures thereof.

45. The method of claim 1 wherein the composition has a pH of about 2 to less than about 5.

46. The method of claim 1 wherein the composition further comprises a hydrotrope in amount of about 0.1% to about 30%, by weight.

47. The method of claim 1 wherein the composition further comprises about 0.1% to about 50% of a polyhydric solvent selected from the group consisting of a diol, a triol, and mixtures thereof.

48. The method of claim 7 wherein the skin has a log reduction against a norovirus of at least 2 about four hours after contact with the composition.

49. The method of claim 7 wherein the skin has a log reduction against a norovirus of at least 2 about eight hours after contact with the composition.

50. The method of claim 8 wherein the skin of the mammal has a skin pH of less than 4 four hours after contact.

51. The method of claim 1 wherein the composition is applied to the surface prior to the surface being exposed to a norovirus.

52. The method of claim 1 wherein the composition is applied multiple times within a twenty-four hour period.

53. The method of claim 2 wherein an effective amount of the organic acid remains in the barrier layer on the surface after ten rinsings with water.

54. The method of claim 1 wherein at least 50%, by weight, of the nonvolatile components of the composition are present on the surface after three rinses with water.

55. A method of inactivating noroviruses and killing bacteria comprising topically applying a composition to an animate or inanimate surface in need of such treatment, said composition comprising:
(a) about 25% to about 95%, by weight, of a disinfecting alcohol;
(b) a virucidally effective amount of an organic acid;
(c) about 0% to about 5%, by weight, of an active antimicrobial agent;
(d) 0% to about 5%, by weight, of a gelling agent; and
(e) water,
wherein the composition has a pH of about 5 or less at 25° C.

56. The method of claim 55 wherein a persistent efficacy against norovirus is imparted to the surface.

57. The method of claim 55 wherein the noroviruses are inactivated for up to about six hours.

58. A method of protecting an individual against infection by noroviruses comprising of applying a composition to hands of the individual in an amount sufficient to eradicate noroviruses,
said composition comprising
(a) about 25% to about 95%, by weight, of a disinfecting alcohol;
(b) a virucidally effective amount of an organic acid;
(c) about 0% to about 5%, by weight, of an active antimicrobial agent;
(d) 0% to about 5%, by weight, of a gelling agent; and
(e) water,
wherein the composition has a pH of about 5 or less at 25° C.

59. The method of claim 58 wherein the composition is applied prior to the individual being exposed to noroviruses.

60. The method of claim 58 wherein the composition is applied multiple times within a twenty-four hour period.

61. The method of claim 58 wherein the composition is rinsed from the hands.

62. The method of claim 58 wherein the composition is allowed to dry and remain on the hands.
